(19) United States
(12) Patent Application Publication
(10) Pub. No.: US 2017/0330029 A1
(43) Pub. Date: Nov. 16, 2017

(54) COMPUTER BASED CONVOLUTIONAL PROCESSING FOR IMAGE ANALYSIS

(71) Applicant: Affectiva, Inc., Boston, MA (US)

(72) Inventors: Panu James Turcot, Pacifica, CA (US); Rana el Kaliouby, Milton, MA (US); Daniel McDuff, Cambridge, MA (US)

(21) Appl. No.: 15/666,048

(22) Filed: Aug. 1, 2017

Related U.S. Application Data
(63) Continuation-in-part of application No. 15/395,750, filed on Dec. 30, 2016.
Application No. 15/395,750 is a continuation-in-part of application No. 15/262,197, filed on Sep. 12, 2016.
Application No. 15/262,197 is a continuation-in-part of application No. 14/796,419, filed on Jul. 10, 2015.
Application No. 14/796,419 is a continuation-in-part of application No. 13/153,745, filed on Jun. 6, 2011, now abandoned.
Application No. 14/796,419 is a continuation-in-part of application No. 14/460,915, filed on Aug. 15, 2014.
Application No. 14/460,915 is a continuation-in-part of application No. 13/153,745, filed on Jun. 6, 2011, now abandoned.
(60) Provisional application No. 62/370,421, filed on Aug. 3, 2016.
(60) Provisional application No. 62/439,928, filed on Dec. 29, 2016.
(60) Provisional application No. 62/442,325, filed on Jan. 4, 2017.
(60) Provisional application No. 62/448,448, filed on Jan. 20, 2017.
(60) Provisional application No. 62/442,291, filed on Jan. 4, 2017.
(60) Provisional application No. 62/469,591, filed on Mar. 10, 2017.
(60) Provisional application No. 62/503,485, filed on May 9, 2017.
(60) Provisional application No. 62/524,606, filed on Jun. 25, 2017.
(60) Provisional application No. 62/273,896, filed on Dec. 31, 2015.
(60) Provisional application No. 62/301,558, filed on Feb. 29, 2016.
(60) Provisional application No. 62/370,421, filed on Aug. 3, 2016.
(60) Provisional application No. 62/217,872, filed on Sep. 12, 2015.
(60) Provisional application No. 62/222,518, filed on Sep. 23, 2015.
(60) Provisional application No. 62/265,937, filed on Dec. 10, 2015.
(60) Provisional application No. 62/273,896, filed on Dec. 31, 2015.
(60) Provisional application No. 62/301,558, filed on Feb. 29, 2016.
(60) Provisional application No. 62/370,421, filed on Aug. 3, 2016.
(60) Provisional application No. 62/023,800, filed on Jul. 11, 2014.
(60) Provisional application No. 62/047,508, filed on Sep. 8, 2014.
(60) Provisional application No. 62/082,579, filed on Nov. 20, 2014.
(60) Provisional application No. 62/128,974, filed on Mar. 5, 2015.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/867,007, filed on Aug. 16, 2013.
(60) Provisional application No. 61/924,252, filed on Jan. 7, 2014.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.

(51) Int. Cl. G06K 9/00; A61B 5/1171; A61B 5/16; A61B 5/00; G06N 3/04; G06K 9/62; G06K 9/66; G06Q 30/02; G06N 7/00

(52) U.S. Cl. CPC G06K 9/00308; G06K 9/6227; A61B 5/1176; A61B 5/165; A61B 5/7267; G06N 3/049; G06K 9/66; G06K 9/6263; A61B 5/0077; G06K 9/00281; A61B 2576/00; G06N 7/005; G06Q 30/0201; G06Q 30/0241

(57) ABSTRACT

Disclosed embodiments provide for deep convolutional computing image analysis. The convolutional computing is accomplished using a multilayered analysis engine. The multilayered analysis engine includes a deep learning network using a convolutional neural network (CNN). The multilayered analysis engine is used to analyze multiple images in a supervised or unsupervised learning process. The multilayered engine is provided multiple images, and the multilayered analysis engine is trained with those images. A subject image is then evaluated by the multilayered analysis engine by analyzing pixels within the subject image to identify a facial portion and identifying a facial expression based on the facial portion. Mental states are inferred using the deep convolutional computer multilayered analysis engine based on the facial expression.

### RELATED APPLICATIONS

This application claims the benefit of U.S. provisional patent applications “Deep Convolutional Neural Network Analysis of Images for Mental States” Ser. No. 62/370,421, filed Aug. 3, 2016, “Image Analysis Framework using Remote Learning with Deployable Artifact” Ser. No. 62/439,928, filed Dec. 29, 2016, “Audio Analysis Learning using Video Data” Ser. No. 62/442,325, filed Jan. 4, 2017, “Vehicle Manipulation using Occupant Image Analysis” Ser. No. 62/448,448, filed Jan. 20, 2017, “Smart Toy Interaction using Image Analysis” Ser. No. 62/442,291, filed Jan. 4, 2017, “Image Analysis for Two-sided Data Hub” Ser. No. 62/469,591, filed Mar. 10, 2017, “Vehicle Artificial Intelligence Evaluation of Mental States” Ser. No. 62/503,485, filed May 9, 2017, and “Image Analysis for Emotional Metric Generation” Ser. No. 62/524,606, filed Jun. 25, 2017.

This application is also a continuation-in-part of U.S. patent application “Image Analysis using Sub-sectional Component Evaluation to Augment Classifier Usage” Ser. No. 15/395,750, filed Dec. 30, 2016, which claims the benefit of U.S. provisional patent applications “Image Analysis Using Sub-Sectional Component Evaluation to Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016.

The patent application “Image Analysis using Sub-sectional Component Evaluation to Augment Classifier Usage” Ser. No. 15/395,750, filed Dec. 30, 2016, is also a continuation-in-part of U.S. patent application “Mental State Event Signature Usage” Ser. No. 15/262,197, filed Sep. 12, 2016, which claims the benefit of U.S. provisional patent applications “Mental State Event Signature Usage” Ser. No. 62/217,872, filed Sep. 12, 2015, “Image Analysis In Support of Robotic Manipulation” Ser. No. 62/222,518, filed Sep. 23, 2015, “Analysis of Image Content with Associated Manipulation of Expression Presentation” Ser. No. 62/265,937, filed Dec. 10, 2015, “Image Analysis Using Sub-Sectional Component Evaluation To Augment Classifier Usage” Ser. No. 62/273,896, filed Dec. 31, 2015, “Analytics for Live Streaming Based on Image Analysis within a Shared Digital Environment” Ser. No. 62/301,558, filed Feb. 29, 2016, and “Deep Convolutional Neural Network Analysis of Images for Mental States” Ser. No. 62/370,421, filed Aug. 3, 2016.

The patent application “Mental State Event Signature Usage” Ser. No. 15/262,197, filed Sep. 12, 2016, is also a continuation-in-part of U.S. patent application “Mental State Event Definition Generation” Ser. No. 14/796,419, filed Jul. 10, 2015, which claims the benefit of U.S. provisional patent applications “Mental State Event Definition Generation” Ser. No. 62/023,800, filed Jul. 11, 2014, “Facial Tracking with Classifiers” Ser. No. 62/047,508, filed Sep. 8, 2014, “Semiconductor Based Mental State Analysis” Ser. No. 62/082,579, filed Nov. 20, 2014, and “Viewership Analysis Based On Facial Evaluation” Ser. No. 62/128,974, filed Mar. 5, 2015. The patent application “Mental State Event Definition Generation” Ser. No. 14/796,419, filed Jul. 10, 2015 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

The patent application “Mental State Event Definition Generation” Ser. No. 14/796,419, filed Jul. 10, 2015 is also a continuation-in-part of U.S. patent application “Mental State Analysis Using an Application Programming Interface” Ser. No. 14/460,915, Aug. 15, 2014, which claims the benefit of U.S. provisional patent applications “Application Programming Interface for Mental State Analysis” Ser. No. 61/867,007, filed Aug. 16, 2013, “Mental State Analysis Using an Application Programming Interface” Ser. No. 61/924,252, filed Jan. 7, 2014, “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014. The patent application “Mental State Analysis Using an Application Programming Interface” Ser. No. 14/460,915, Aug. 15, 2014 is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011.

Each of the foregoing applications is hereby incorporated by reference in its entirety.

### FIELD OF ART

This application relates generally to image analysis and more particularly to computer based convolutional processing for image analysis.

### BACKGROUND

Human emotions often result in facial expressions. The human face contains over forty muscles acting in coordination to produce numerous facial expressions. The facial expressions can represent emotions such as anger, fear, sadness, disgust, contempt, surprise, and happiness. Facial muscles cause expressions by brow raising, smiling, nose wrinkling, and other actions that are indicative of emotions or reactions to an external stimulus. For example, a person might wrinkle his nose in response to an unpleasant smell, smile in response to something he finds funny, and lower his brow in response to something invoking confusion or skepticism.

On any given day, an individual is confronted with a wide variety of external stimuli. The stimuli can be any combination of visual, aural, tactile, and other types of stimuli, and, alone or in combination, can invoke strong emotions in the individual. An individual's reactions to received stimuli provide insight into the thoughts and feelings of the individual. Furthermore, the individual's responses to the stimuli can have a profound impact on the mental states experienced by the individual. The mental states of an individual can vary widely, ranging from happiness to sadness, contentedness to worry, and calm to excitement, to name only a very few possible states.

The level of strength of the emotion or mental state experienced may be reflected in the level or intensity of a facial expression. For example, there are multiple levels of smile that a person can make in response to internal or external stimuli. A low intensity smile may include lips being closed, with a slight upward rise at the corners of the mouth. A medium intensity smile may include more rise at the corners of the mouth and showing some of the front teeth. A high intensity smile may include even more rise at the corners of the mouth and showing additional front teeth. Eyebrows and other facial features also vary with intensity of the smile. Similar to smiles, other facial expressions can have multiple levels, each reflecting a level or intensity of an emotion. For example, fear can be portrayed by the raising of the upper eyelids, contraction of the lower eyelids, and muscle contractions to pull the eyebrows up and in. The amount of movement in these regions of the face can correlate to the level of fear being experienced.

Different people may respond differently to a given stimulus. For example, some people may smile when afraid or nervous. Thus, there can be a difference between a facial expression and an underlying mental state. The smile a person produces when nervous may be different than the smile they produce when happy. Mental or emotional states can play a role in how people interpret external stimuli. Emotions such as happiness, sadness, fear, laughter, relief, angst, worry, anguish, anger, regret, and frustration are often reflected in facial expressions. Thus, the study of facial expressions and their meanings can provide important insight into human behavior.

### SUMMARY

Disclosed embodiments provide capabilities for image analysis using a convolutional-processing-initialized computer, along with techniques for training and using the system. The system includes a multilayered analysis engine. The multilayered analysis engine includes a deep learning network using a convolutional neural network (CNN). The multilayered analysis engine is used to analyze a plurality of images in a supervised or unsupervised learning process. Utilizing one or more imaging devices, the arrangement obtains a plurality of images used to train the multilayered analysis engine. Then a subject image is evaluated by the multilayered analysis engine through analyzing pixels within the subject image to identify a facial portion, and identifying a facial expression based on the facial portion. Mental states can then be inferred from the facial expression. A computer-implemented method for image analysis is disclosed comprising: initializing a computer for convolutional processing; obtaining, using an imaging device, a plurality of images; training, on the computer initialized for convolutional processing, a multilayered analysis engine using the plurality of images, wherein the multilayered analysis engine includes multiple layers that include one or more convolutional layers and one or more hidden layers, and wherein the multilayered analysis engine is used for emotional analysis; and evaluating a further image using the multilayered analysis engine wherein the evaluating includes: analyzing pixels within the further image to identify a facial portion; and identifying a facial expression based on the facial portion. Mental states can be inferred based on emotional content within a face associated with the facial portion. A facial expression can be identified using a hidden layer from the one or more hidden layers. The multilayered analysis engine can include a max pooling layer. Weights can be updated during a backpropagation process through the multilayered analysis engine. The training of the multilayered analysis engine can comprise deep learning.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram representing deep convolutional processing image analysis.

FIG. 2 is a flow diagram representing training.

FIG. 3 is an example showing a pipeline for facial analysis layers.

FIG. 4 is an example illustrating a deep network for facial expression parsing.

FIG. 5 is an example illustrating a convolution neural network.

FIG. 6 is a diagram showing image collection including multiple mobile devices.

FIG. 7 illustrates feature extraction for multiple faces.

FIG. 8 shows live streaming of social video.

FIG. 9 shows example facial data collection including landmarks.

FIG. 10 shows example facial data collection including regions.

FIG. 11 is a flow diagram for detecting facial expressions.

FIG. 12 is a flow diagram for the large-scale clustering of facial events.

FIG. 13 shows unsupervised clustering of features and characterizations of cluster profiles.

FIG. 14A shows example tags embedded in a webpage.

FIG. 14B shows invoking tags to collect images.

FIG. 15 is a system diagram for image analysis.

### DETAILED DESCRIPTION

Emotion analysis is a very complex task. Understanding and evaluating moods, emotions, or mental states requires a nuanced evaluation of facial expressions or other cues generated by people. Techniques for image analysis, and resulting mental state analysis, using a multilayered analysis engine are described herein. Image analysis is a critical element in mental state analysis. Mental state analysis is important in many areas. The understanding of mental states can be used in a variety of fields, such as improving marketing analysis, assessing the effectiveness of customer service and retail experiences, and evaluating the consumption of content such as movies and videos. For example, identifying points of frustration in a customer transaction can allow a company to take action to address the causes of the frustration. By streamlining processes, key performance areas such as customer satisfaction and customer transaction throughput can be improved, resulting in increased sales and revenues. In a content scenario, producing compelling content that achieves the desired effect (e.g. fear, shock, laughter, etc.) can result in increased ticket sales and/or increased advertising revenue. For example, if a movie studio is producing a horror movie, it is desirable to know if the scary scenes in the movie are achieving the desired effect. By conducting tests in sample audiences, and analyzing faces in the audience, a computer-implemented method and system can process thousands of faces to assess the overall mental state at the time of the scary scenes. In some ways, such an analysis can be more effective than surveys that ask audience members questions, since audience members may consciously or subconsciously change answers based on peer pressure or other factors. However, spontaneous facial expressions can be more difficult to conceal. Thus, by analyzing facial expressions en masse, important information regarding the mental state of the audience can be obtained.

In embodiments, a multilayered analysis engine including a convolutional neural network is used to analyze multiple faces. The faces can be tagged to indicate an expression and/or mental state. The tagging can be performed outside of the convolutional neural network. Thus, in a supervised learning scenario, images are input to the multilayered analysis engine and the multilayered analysis engine is trained to recognize one or more facial expressions and/or mental states. The output of the multilayered analysis engine can be reviewed for effectiveness. Weights between the layers can be adjusted to further refine the multilayered analysis engine for improved reliability in terms of analyzing facial expressions and/or mental states. In embodiments, an input layer performs image preprocessing functions including, but not limited to, identifying face boundaries, identifying face landmarks, and extracting facial features. Additional image preprocessing functions can include, but are not limited to, rotating the face, cropping the image and/or establishing a bounding box as a constraint for subsequent layers, and performing contrast, saturation, and/or hue adjustments. Additional image preprocessing such as edge detection, spatial filtering, and frequency domain filtering can also be performed. The output of the system includes data indicative of a facial expression and/or mental state. Once trained, the system can analyze many thousands of faces much faster than would be possible for a team of humans to identify just a few faces. As a result, users can quickly obtain important feedback for business situations such as customer satisfaction, consumption of media content, and effectiveness of advertisements. It should be understood that such a multilayered analysis engine, or something similar, could also be utilized for emotion analysis of verbal information.

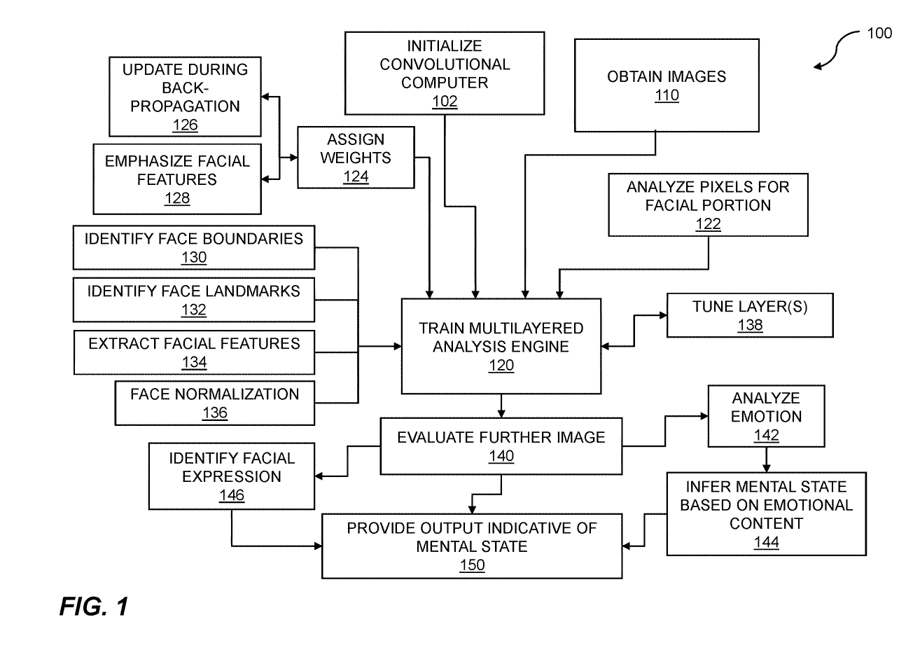

Referring now to the figures, FIG. 1 is a flow diagram representing deep convolutional processing image analysis. The flow 100 shows a computer-implemented method for image analysis. The flow 100 includes initializing a convolutional computer 102. A convolutional computer can either be specialized hardware designed specifically for neural network convolution, or it can comprise unique software that enables a generic computer to operate as a specialized convolutional machine. The convolutional computer may exist as dedicated hardware, or it may exist as part of a networked structure, such as a supercomputer, a supercomputer cluster, a cloud-based system, a server-based system, a distributed computer network, and the like. The flow 100 includes obtaining a plurality of images 110. Each of the plurality of images can include at least one human face. In embodiments, each image includes metadata. The metadata can include information about each face that is entered by human coders. The metadata can include a perceived facial expression and/or mental state. The metadata can further include demographic information such as an age range, gender, and/or ethnicity information. Since different demographic groups might register emotions in different ways, the demographic information can be used to further enhance the output results of the multilayered analysis engine. For example, some demographic groups might not smile as frequently or as intensely as others. Thus, a compensation can be applied in these circumstances when analyzing smiles to effectively normalize the results. A similar approach can be applied to other facial expressions and/or mental states.

The flow 100 includes training a multilayered analysis engine 120 using the plurality of images, wherein the multilayered analysis engine includes multiple layers that include one or more convolutional layers and one or more hidden layers, and wherein the multilayered analysis engine is used for emotional analysis. Hidden layers are layers within the multilayered analysis engine with outputs that are not externally exposed. The output of hidden layers feeds another layer, but the output of the hidden layer is not directly observable. The training can include submitting multiple images to the multilayered analysis engine. In a supervised learning scenario, the multilayered analysis engine makes an assessment of the facial expression and/or mental state and compares its output to the human-coded assessment in the metadata. Various parameters such as weights between the layers can be adjusted until a majority of the input images are correctly classified by the multilayered analysis engine. Alternate embodiments can be implemented wherein stopping criteria is used. A desired target or accuracy is selected and images are analyzed that have been labelled. Once the target mental states or facial expressions identified by the neural network match those labelled then the learning can be stopped. In some cases, stopping criteria can be the number of images which are matched, the number of re-learning steps used, error rates stop decreasing, error rates only reduce by a limited predefined amount, the number of back propagation operations performed, and so on.

Thus, the flow 100 can further include assigning weights 124. The assignment of weights can be influenced by updating during backpropagation 126. Back propagation can include calculation of a loss function gradient which is used to update the values of the weights as part of a supervised learning process. The assignment of weights can be selected to emphasize facial features 128, such as eyes, mouth, nose, eyelids, eyebrows, and/or chin. In other embodiments, the input images are not associated with metadata pertaining to facial expressions and/or mental states. In such cases, the multilayered analysis engine is trained using an unsupervised learning process.

The flow 100 includes evaluating a further image 140 using the multilayered analysis engine, wherein the evaluating includes analyzing pixels within the further image to identify a facial portion 122 and identifying a facial expression 146 based on the facial portion. The facial expression can include a smile, frown, laugh, expression of surprise, concern, confusion, and/or anger, among others. The analyzing of pixels for identifying a facial portion 122 can include identifying a face contour, as well as locating facial features such as eyes, nose, mouth, chin, and cheekbones. The further image 140 is a subject image that is to be analyzed by the multilayered analysis engine. Thus, once the multilayered analysis engine is trained, a subject image can be input to the multilayered analysis engine, and the multilayered analysis engine can analyze the subject image to determine a facial expression 146 and/or an output indicative of a mental state 150. A facial expression can correlate to more than one mental state, depending on the circumstances. For example, a smile can indicate happiness in many situations. However, in some cases, a person might smile while experiencing another mental state, like embarrassment. The “happy” smile might have slightly different attributes than the “embarrassed” smile. For example, the lip corners can be pulled higher in a “happy” smile than in an “embarrassed” smile. Through the training of the multilayered analysis engine 120, the multilayered analysis engine can learn the difference between the variants of a facial expression (e.g. smiles) to provide an output indicative of mental state 150.

The flow 100 includes analyzing an emotion 142. The emotion can be a representation of how the subject person is feeling at the time of image acquisition. The emotion analysis can be based on facial features and can include the use of action units (AUs). Such AUs can include, but are not limited to, brow lowerer, nose wrinkler, and mouth stretch, just to name a few. In practice, many more AUs can be examined during analyzing an emotion 142. The flow 100 includes inferring a mental state based on emotional content within a face associated with the facial portion 144. The emotional content can include, but is not limited to, facial expressions such as smiles, smirks, and frowns. Emotional content can also include actions such as lip biting, eye shifting, and head tilting. Furthermore, external features such as tears on a face can be part of the emotional content. For example, detecting the presence of tears can be used in determining an expression/mental state of sorrow. However, in some instances, tears can also signify a mental state of extreme joy. The multilayered analysis engine can examine other factors in conjunction with the presence of tears to distinguish between the expressions of sorrow and joy.

The flow 100 includes tuning one or more layers 138 within the multiple layers for a particular mental state. In some embodiments, the tuning is for the last layer within the multiple layers where that last layer is tuned for identifying a particular mental state. In other embodiments, multiple layers are tuned. The multilayered analysis engine can include many layers. In embodiments, tuning the last layer 138 is used to adjust the output so that the mental state and/or facial expressions provided by the multilayered analysis engine agree with the images used to train the multilayered analysis engine. For example, if images used for training contain facial expressions indicative of joy, but the output provided by the multilayered analysis engine is not indicating joy in a majority of the cases, then the last layer 138 can be tuned to make the output provided by the multilayered analysis engine indicate joy in a majority of the cases. The tuning can include adjusting weights, constants, or functions within and/or input to the last layer. Furthermore, other tuning techniques can be employed including learning from previous layers. In addition, later layers can be tuned to learn different expression or further expressions so that other mental states or facial expressions are identified by the neural network.

The flow 100 includes identifying boundaries of the face 130. Identifying the existence of a face within an image can be accomplished in a variety of ways, including, but not limited to, utilizing a histogram-of-oriented-gradient (HoG) based object detectors. The flow 100 includes identifying landmarks of the face 132. The landmarks are points of interest within a face. These can include, but are not limited to, the right eye, left eye, nose base, and lip corners. The flow 100 includes extracting features of the face 134. The features can include, but are not limited to, eyes, eyebrows, eyelids, lips, lip corners, chin, cheeks, teeth, and dimples.

The flow 100 can include various types of face normalizing 136 including rotating, resizing, contrast adjustment, brightness adjustment, cropping, and so on. One or more of these normalization processes can be executed on faces within the plurality of images. The normalization steps can be performed on images, videos, or frames within a video. In embodiments, the image is rotated to a fixed orientation by an input layer of the multilayered analysis engine. For example, a face that is tilted at a 30-degree angle can be rotated such that it is oriented vertically, so that the mouth is directly below the nose of the face. In this way, the subsequent layers of the multilayered analysis engine work with a consistent image orientation.

As part of the training, the flow 100 includes training an emotion classifier for emotional content. The emotion classifier can include one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, shock, surprise, fear, curiosity, humor, sadness, poignancy, or mirth. In embodiments, the multilayered analysis engine is trained for a specific emotion such as shock. For example, in an application for determining the effectiveness of scary scenes in a horror movie, the multilayered analysis engine can be trained specifically to identify a facial expression of shock, corresponding to a mental state of surprise combined with fear. The horror movie is then shown to a test audience, where one or more cameras obtain images of the audience as the movie is being viewed. Facial images are acquired at a predetermined time after the presentation of a scary scene. For example, the images can be acquired at a time ranging from about 300 milliseconds to about 700 milliseconds after presentation of the scary scene. This allows a viewer sufficient processing time to react to the scene, but is not so long that the viewer is no longer expressing their initial reaction.

In the flow 100, the training of the multilayered analysis engine comprises deep learning. Deep learning is a type of machine learning utilizing neural networks. In general, it is non-trivial for a computer to interpret the meaning of raw sensory input data, such as digital images that are represented as an array of pixels. Converting from an array or subset of pixels to identification of an object within the image, such as a human face, is very complicated. Direct evaluation of this mapping is computationally impractical to solve directly. However, embodiments disclosed herein comprise a multilayered analysis engine that utilizes deep learning. The multilayered analysis engine can determine features within an image by dividing the highly complex mapping into a series of more simple mappings, each processed by a different layer of the multilayered analysis engine. The input image is presented to an input layer, which performs initial processing on the image. Then one or more hidden layers extract features from the image. In embodiments, the outputs of the hidden layers are not directly observable. The hidden layers can provide evaluation of mental states or facial expressions without specific interpretation or labels being provided. The outputs of the hidden layers can, however, be used by further layers within the convolutional neural network to perform the mental state or facial expression analysis.

When an image is input to the multilayered analysis engine, the input layer can be used to identify edges by comparing the brightness of neighboring pixels or other edge detection process. The edges can then be input to a subsequent hidden layer, which can then extract features such as corners. The process continues with additional hidden layers, each additional layer performing additional operations, and culminating with an output layer that produces a result which includes a facial expression and/or mental state. Thus, the deep learning network provides an improved automated detection of facial expressions and/or mental states, enabling new and exciting applications such as large-scale evaluation of emotional response.

In the flow 100, the multilayered analysis engine comprises a convolutional neural network. Convolutional neural networks (CNNs) share many properties with ordinary neural networks. For example, they both include neurons that have learnable weights and biases. Each node/neuron receives some inputs and performs a function that determines if the node/neuron “fires” and generates an output. However, CNNs are well-suited for inputs that are images, allowing for certain optimizations to be incorporated into the architecture of the CNN. These then make the forward function more efficient to implement and improve the performance regarding image analysis. In the flow 100, the evaluation of emotional content of the face includes detection of one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, sadness, poignancy, or mirth. Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

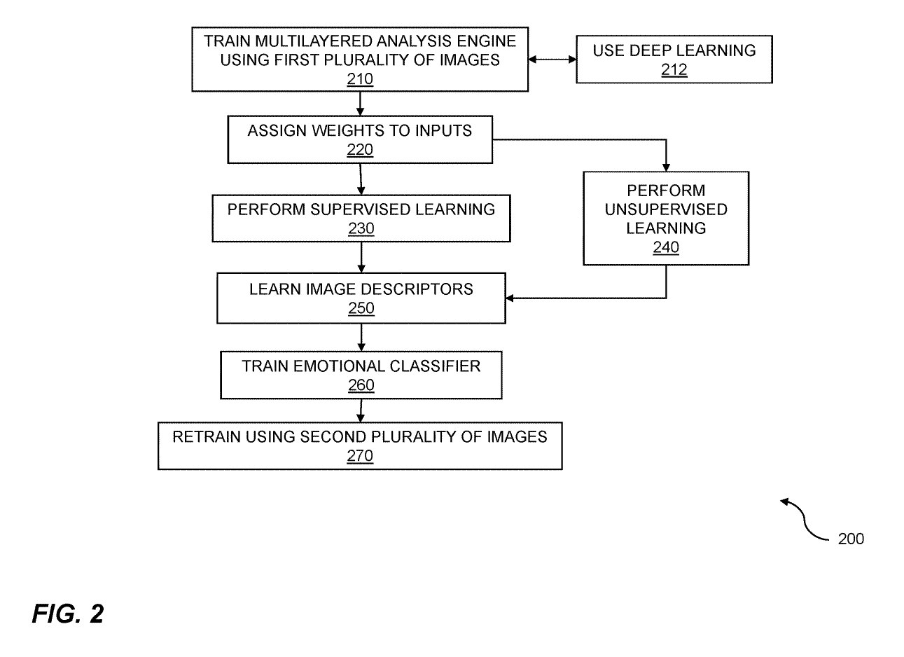

FIG. 2 is a flow diagram representing training. The flow 200 includes training a multilayered analysis engine using a first plurality of images 210. The flow 200 includes assigning weights to inputs 220. The weights can be applied to inputs to the layers that comprise the multilayered analysis engine. In some embodiments, the weights are assigned an initial value that update during the training of the multilayered analysis engine, based on processes such as backpropagation. In embodiments, the flow 200 includes performing supervised learning 230 as part of the training by using a set of images, from the plurality of images, that have been labeled for mental states. In other embodiments, the flow 200 includes performing unsupervised learning 240 as part of the training.

As part of the training, the flow 200 includes learning image descriptors 250 for emotional content. The image descriptors can include features within an image such as those represented by action units (AU). The descriptors can include, but are not limited to, features such as a raised eyebrow, a wink of one eye, or a smirk. In the flow 200, the image descriptors are identified based on a temporal co-occurrence with an external stimulus. The external stimulus can include media content such as an advertisement, a scene from a movie, or an audio clip. Additionally, the external stimulus can include a live event happening in the room where the subject is, such as a siren, a thunder clap, or a flashing light. The flow 200 includes training emotional classifiers 260. By analyzing multiple training images, the multilayered analysis engine can learn that lip corners pulled down in conjunction with lowered brows may be indicative of a mental state of disappointment. As more and more images are reviewed, the multilayered analysis engine generally becomes better at analysis of mental state and/or facial expressions.

The flow 200 includes re-training the multilayered analysis engine using a second plurality of images 270. In some embodiments, once an initial training session has completed, the retraining occurs using images of a specific subset of emotions. For example, the second plurality of images can focus exclusively on fear, shock, and surprise. The second plurality of images can be tailored to the emotions of interest for the users of the multilayered analysis engine. In the flow 200, the re-training updates weights on a subset of layers within the multilayered analysis engine. In embodiments, the subset of layers is a single layer within the multilayered analysis engine. Additionally, the flow 200 can include the use of deep learning 212 to accomplish the training. Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors.

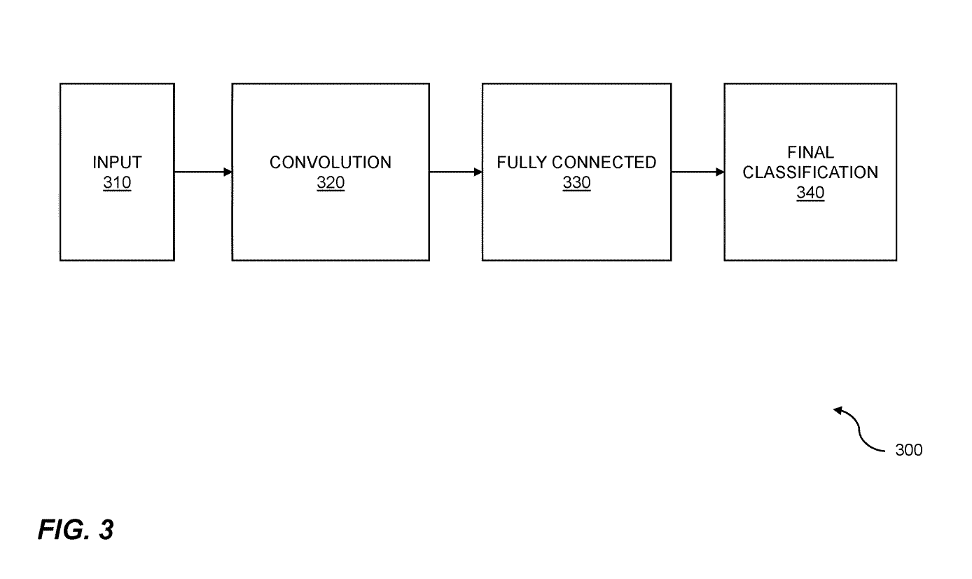

FIG. 3 is an example 300 showing a pipeline for facial analysis layers. The example 300 includes an input layer 310. The input layer 310 receives image data. The image data can be input in a variety of formats, such as JPEG, TIFF, BMP, and GIF. Compressed image formats can be decompressed into arrays of pixels, wherein each pixel can include an RGB tuple. The input layer 310 can then perform processing such as identifying boundaries of the face, identifying landmarks of the face, extracting features of the face, and/or rotating a face within the plurality of images. The output of the input layer can then be input to a convolution layer 320. The convolution layer 320 can represent a convolutional neural network and can contain a plurality of hidden layers within it. A layer from the multiple layers can be fully connected. The convolutional layer 320 can reduce the amount of data feeding into a fully connected layer 330. The fully connected layer processes each pixel/data point from the convolutional layer 320. A last layer within the multiple layers can provide output indicative of a certain mental state. The last layer is the final classification layer 340. The output of the final classification layer 340 can be indicative of the mental states of faces within the images that are provided to input layer 310.

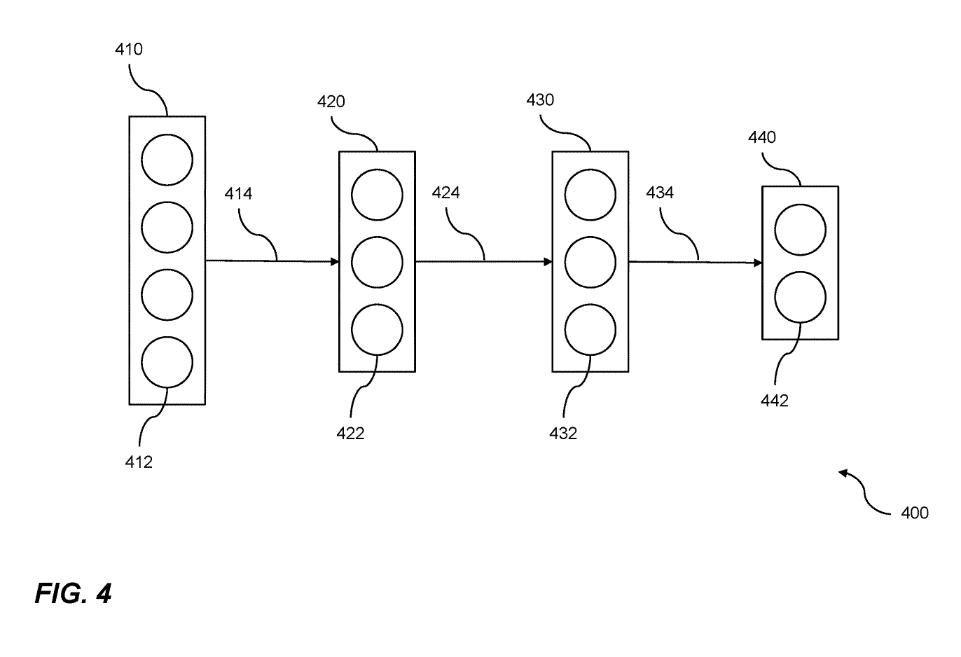

FIG. 4 is an example 400 illustrating a deep network for facial expression parsing. A first layer 410 of the deep network is comprised of a plurality of nodes 412. Each of nodes 412 serves as a neuron within a neural network. The first layer can receive data from an input layer (e.g. 310 of FIG. 3). The output of the first layer 410 feeds to a layer 420. The layer 420 further comprises a plurality of nodes 422. A weight 414 adjusts the output of the first layer 410 which is being input to the layer 420. In embodiments, the layer 420 is a hidden layer. The output of the layer 420 feeds to a layer 430. The layer 430 further comprises a plurality of nodes 432. A weight 424 adjusts the output of the layer 420 which is being input to the layer 430. In embodiments, the layer 430 is also a hidden layer. The output of the layer 430 feeds to a layer 440. The layer 440 further comprises a plurality of nodes 442. A weight 434 adjusts the output of the layer 430 which is being input to the layer 440. The layer 440 can be a final layer, providing a facial expression and/or mental state as its output. The facial expression can be identified using a hidden layer from the one or more hidden layers. The weights can be provided on inputs to the multiple layers to emphasize certain facial features within the face. The training can comprise assigning weights to inputs on one or more layers within the multilayered analysis engine. In embodiments, one or more of the weights (414, 424, and/or 434) can be adjusted or updated during training. The assigning weights can be accomplished during a feed-forward pass through the multilayered analysis engine. In a feed-forward arrangement, the information moves forward from the input nodes through the hidden nodes and on to the output nodes. Additionally, the weights can be updated during a backpropagation process through the multilayered analysis engine.

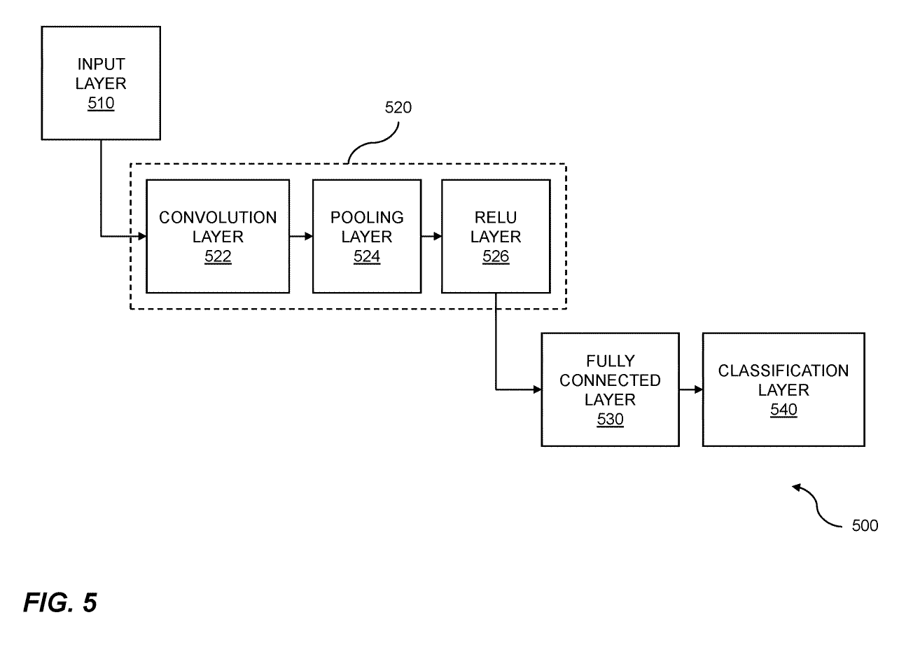

FIG. 5 is an example 500 illustrating a convolution neural network. The network includes an input layer 510 which receives image data. The image data can be input in a variety of formats, such as JPEG, TIFF, BMP, and GIF. Compressed image formats can be decompressed into arrays of pixels, wherein each pixel can include an RGB tuple. The input layer 510 can then perform processing such as identifying boundaries of the face, identifying landmarks of the face, extracting features of the face, and/or rotating a face within the plurality of images.

The network includes a collection of intermediate layers 520. The multilayered analysis engine can include a convolutional neural network. Thus, the intermediate layers can include a convolution layer 522. The convolution layer 522 can include multiple sublayers, including hidden layers within it. The output of the convolution layer 522 feeds into a pooling layer 524. The pooling layer 524 performs a data reduction, which makes the overall computation more efficient. Thus the pooling layer reduces the spatial size of the image representation to reduce the amount of parameters and computation in the network. In some embodiments, the pooling layer is implemented using filters of size 2×2, applied with a stride of two samples for every depth slice along both width and height, resulting in a reduction of 75-percent of the downstream node activations. The multilayered analysis engine can further include a max pooling layer 524. Thus, in embodiments, the pooling layer is a max pooling layer in which the output of the filters is based on a maximum of the inputs. For example, with a 2×2 filter, the output is based on a maximum value from the four input values. In other embodiments, the pooling layer is an average pooling layer or L2-norm pooling layer. Various other pooling schemes are possible.

The intermediate layers can include a Rectified Linear Units (RELU) layer 526. The output of the pooling layer 524 can be input to the RELU layer 526. In embodiments, the RELU layer implements an activation function such as f(x)−max(0,x), thus providing an activation with a threshold at zero. In some embodiments, the RELU layer 526 is a leaky RELU layer. In this case, instead of the activation function providing zero when x<0, a small negative slope is used, resulting in an activation function such as f(x)=1(x<0)(αx)+1(x>=0)(x). This can reduce the risk of “dying RELU” syndrome, where portions of the network can be “dead” with nodes/neurons that do not activate across the training dataset. The image analysis can comprise training a multilayered analysis engine using the plurality of images, wherein the multilayered analysis engine can include multiple layers that include one or more convolutional layers 522 and one or more hidden layers, and wherein the multilayered analysis engine can be used for emotional analysis.

The example 500 includes a fully connected layer 530. The fully connected layer 530 processes each pixel/data point from the output of the collection of intermediate layers 520. The fully connected layer 530 takes all neurons in the previous layer and connects them to every single neuron it has. The output of the fully connected layer 530 provides input to a classification layer 540. The output of the classification layer 540 provides a facial expression and/or mental state as its output. Thus, a multilayered analysis engine such as the one depicted in FIG. 5 processes image data using weights, models the way the human visual cortex performs object recognition and learning, and is effective for analysis of image data to infer facial expressions and mental states.

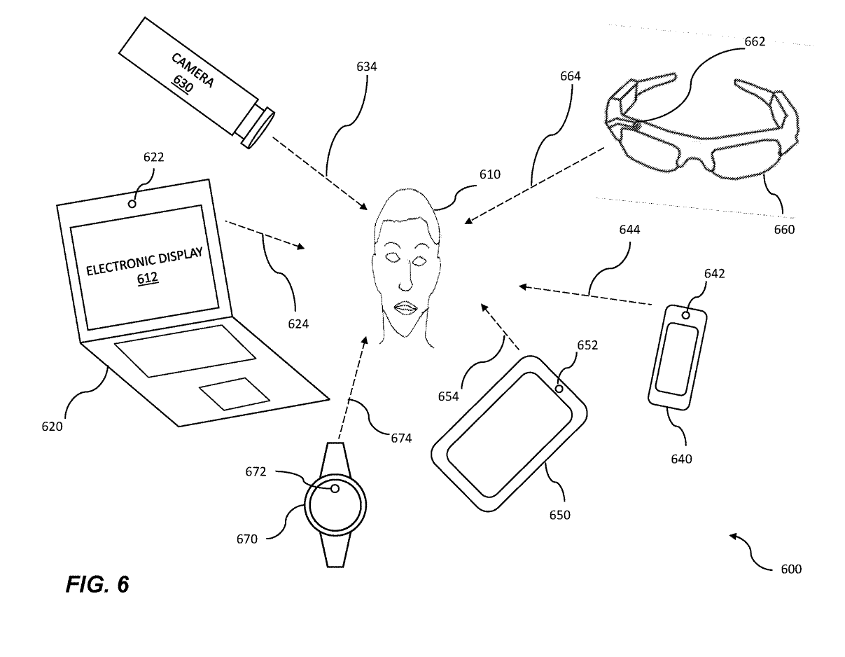

FIG. 6 is a diagram showing image collection including multiple mobile devices. Images from these multiple devices can be used by the convolutional neural net (CNN) to evaluate emotions. The collected images can be analyzed for mental state analysis and/or facial expressions. A plurality of images of an individual viewing an electronic display can be received. A face can be identified in an image, based on the use of image classifiers. The plurality of images can be evaluated to determine mental states and/or facial expressions of the individual. In the diagram 600, the multiple mobile devices can be used singly or together to collect video data on a user 610. While one person is shown, the video data can be collected on multiple people. A user 610 can be observed as she or he is performing a task, experiencing an event, viewing a media presentation, and so on. The user 610 can be shown one or more media presentations, political presentations, or social media, or another form of displayed media. The one or more media presentations can be shown to a plurality of people. The media presentations can be displayed on an electronic display 612 or another display. The data collected on the user 610 or on a plurality of users can be in the form of one or more videos, video frames, still images, etc. The plurality of videos can be of people who are experiencing different situations. Some example situations can include the user or plurality of users being exposed to TV programs, movies, video clips, social media, and other such media. The situations could also include exposure to media such as advertisements, political messages, news programs, and so on. As noted before, video data can be collected on one or more users in substantially identical or different situations and viewing either a single media presentation or a plurality of presentations. The data collected on the user 610 can be analyzed and viewed for a variety of purposes including expression analysis, mental state analysis, and so on. The electronic display 612 can be on a laptop computer 620 as shown, a tablet computer 650, a cell phone 640, a television, a mobile monitor, or any other type of electronic device. In one embodiment, expression data is collected on a mobile device such as a cell phone 640, a tablet computer 650, a laptop computer 620, or a watch 670. Thus, the multiple sources can include at least one mobile device, such as a phone 640 or a tablet 650, or a wearable device such as a watch 670 or glasses 660. A mobile device can include a front-side camera and/or a back-side camera that can be used to collect expression data. Sources of expression data can include a webcam 622, a phone camera 642, a tablet camera 652, a wearable camera 662, and a mobile camera 630. A wearable camera can comprise various camera devices such as the watch camera 672. A mobile device could include an automobile, truck, or other vehicle. The mental state analysis could be performed by such a vehicle or devices and system with which the vehicle communicates.

As the user 610 is monitored, she or he might move due to the nature of the task, boredom, discomfort, distractions, or for another reason. As the user moves, the camera with a view of the user's face can be changed. Thus, as an example, if the user 610 is looking in a first direction, the line of sight 624 from the webcam 622 is able to observe the user's face, but if the user is looking in a second direction, the line of sight 634 from the mobile camera 630 is able to observe the user's face. Furthermore, in other embodiments, if the user is looking in a third direction, the line of sight 644 from the phone camera 642 is able to observe the user's face, and if the user is looking in a fourth direction, the line of sight 654 from the tablet camera 652 is able to observe the user's face. If the user is looking in a fifth direction, the line of sight 664 from the wearable camera 662, which can be a device such as the glasses 660 shown and can be worn by another user or an observer, is able to observe the user's face. If the user is looking in a sixth direction, the line of sight 674 from the wearable watch-type device 670, with a camera 672 included on the device, is able to observe the user's face. In other embodiments, the wearable device is another device, such as an earpiece with a camera, a helmet or hat with a camera, a clip-on camera attached to clothing, or any other type of wearable device with a camera or other sensor for collecting expression data. The user 610 can also use a wearable device including a camera for gathering contextual information and/or collecting expression data on other users. Because the user 610 can move her or his head, the facial data can be collected intermittently when she or he is looking in a direction of a camera. In some cases, multiple people can be included in the view from one or more cameras, and some embodiments include filtering out faces of one or more other people to determine whether the user 610 is looking toward a camera. All or some of the expression data can be continuously or sporadically available from the various devices and other devices. The changes in the direction in which the user 610 is looking or facing can be used in determining mental states associated with a piece of media content.

The captured video data can include facial expressions and can be analyzed on a computing device such as the video capture device or on another separate device. The analysis can take place on one of the mobile devices discussed above, on a local server, on a remote server, and so on. In embodiments, some of the analysis takes place on the mobile device, while other analysis takes place on a server device. The analysis of the video data can include the use of a classifier. The video data can be captured using one of the mobile devices discussed above and sent to a server or another computing device for analysis. However, the captured video data including expressions can also be analyzed on the device which performed the capturing. The analysis can be performed on a mobile device where the videos were obtained with the mobile device and wherein the mobile device includes one or more of a laptop computer, a tablet, a PDA, a smartphone, a wearable device, and so on. In another embodiment, the analyzing comprises using a classifier on a server or another computing device other than the capturing device.

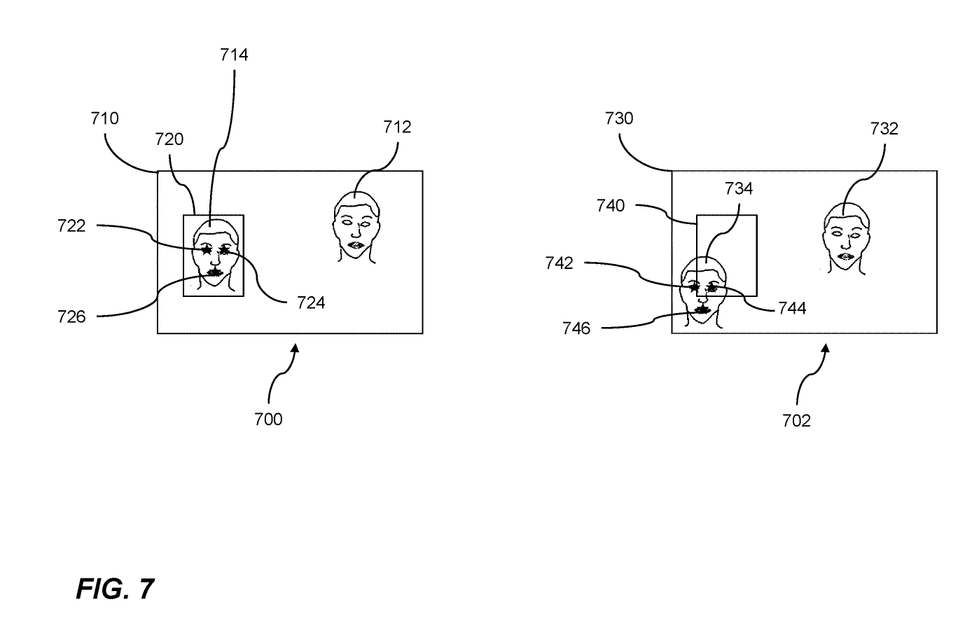

FIG. 7 illustrates feature extraction for multiple faces. The features can be evaluated within a deep learning environment. The feature extraction for multiple faces can be performed for faces that can be detected in multiple images. The images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. The feature extraction can be performed by analysis using one or more processors, using one or more video collection devices, and by using a server. The analysis device can be used to perform face detection for a second face, as well as for facial tracking of the first face. One or more videos can be captured, where the videos contain one or more faces. The video or videos that contain the one or more faces can be partitioned into a plurality of frames, and the frames can be analyzed for the detection of the one or more faces. The analysis of the one or more video frames can be based on one or more classifiers. A classifier can be an algorithm, heuristic, function, or piece of code that can be used to identify into which of a set of categories a new or particular observation, sample, datum, etc. should be placed. The decision to place an observation into a category can be based on training the algorithm or piece of code, by analyzing a known set of data, known as a training set. The training set can include data for which category memberships of the data can be known. The training set can be used as part of a supervised training technique. If a training set is not available, then a clustering technique can be used to group observations into categories. The latter approach, or unsupervised learning, can be based on a measure (i.e. distance) of one or more inherent similarities among the data that is being categorized. When the new observation is received, then the classifier can be used to categorize the new observation. Classifiers can be used for many analysis applications including analysis of one or more faces. The use of classifiers can be the basis of analyzing the one or more faces for gender, ethnicity, and age; for detection of one or more faces in one or more videos; for detection of facial features, for detection of facial landmarks, and so on. The observations can be analyzed based on one or more of a set of quantifiable properties. The properties can be described as features and explanatory variables and can include various data types that can include numerical (integer-valued, real-valued), ordinal, categorical, and so on. Some classifiers can be based on a comparison between an observation and prior observations, as well as based on functions such as a similarity function, a distance function, and so on.

Classification can be based on various types of algorithms, heuristics, codes, procedures, statistics, and so on. Many techniques exist for performing classification. This classification of one or more observations into one or more groups can be based on distributions of the data values, probabilities, and so on. Classifiers can be binary, multiclass, linear, and so on. Algorithms for classification can be implemented using a variety of techniques, including neural networks, kernel estimation, support vector machines, use of quadratic surfaces, and so on. Classification can be used in many application areas such as computer vision, speech and handwriting recognition, and so on. Classification can be used for biometric identification of one or more people in one or more frames of one or more videos.

Returning to FIG. 7, the detection of the first face, the second face, and multiple faces can include identifying facial landmarks, generating a bounding box, and prediction of a bounding box and landmarks for a next frame, where the next frame can be one of a plurality of frames of a video containing faces. A first video frame 700 includes a frame boundary 710, a first face 712, and a second face 714. The video frame 700 also includes a bounding box 720. Facial landmarks can be generated for the first face 712. Face detection can be performed to initialize a second set of locations for a second set of facial landmarks for a second face within the video. Facial landmarks in the video frame 700 can include the facial landmarks 722, 724, and 726. The facial landmarks can include corners of a mouth, corners of eyes, eyebrow corners, the tip of the nose, nostrils, chin, the tips of ears, and so on. The performing of face detection on the second face can include performing facial landmark detection with the first frame from the video for the second face and can include estimating a second rough bounding box for the second face based on the facial landmark detection. The estimating of a second rough bounding box can include the bounding box 720. Bounding boxes can also be estimated for one or more other faces within the boundary 710. The bounding box can be refined, as can one or more facial landmarks. The refining of the second set of locations for the second set of facial landmarks can be based on localized information around the second set of facial landmarks. The bounding box 720 and the facial landmarks 722, 724, and 726 can be used to estimate future locations for the second set of locations for the second set of facial landmarks in a future video frame from the first video frame.

A second video frame 702 is also shown. The second video frame 702 includes a frame boundary 730, a first face 732, and a second face 734. The second video frame 702 also includes a bounding box 740 and the facial landmarks 742, 744, and 746. In other embodiments, multiple facial landmarks are generated and used for facial tracking of the two or more faces of a video frame, such as the shown second video frame 702. Facial points from the first face can be distinguished from other facial points. In embodiments, the other facial points include facial points of one or more other faces. The facial points can correspond to the facial points of the second face. The distinguishing of the facial points of the first face and the facial points of the second face can be used to distinguish between the first face and the second face, to track either or both of the first face and the second face, and so on. Other facial points can correspond to the second face. As mentioned above, multiple facial points can be determined within a frame. One or more of the other facial points that are determined can correspond to a third face. The location of the bounding box 740 can be estimated, where the estimating can be based on the location of the generated bounding box 720 shown in the first video frame 700. The three facial landmarks shown, facial landmarks 742, 744, and 746, might lie within the bounding box 740 or might not lie partially or completely within the bounding box 740. For instance, the second face 734 may move between the first video frame 700 and the second video frame 702. Based on the accuracy of the estimating of the bounding box 740, a new estimation can be determined for a third, future frame from the video, and so on. The evaluation can be performed, all or in part, on semiconductor-based logic.

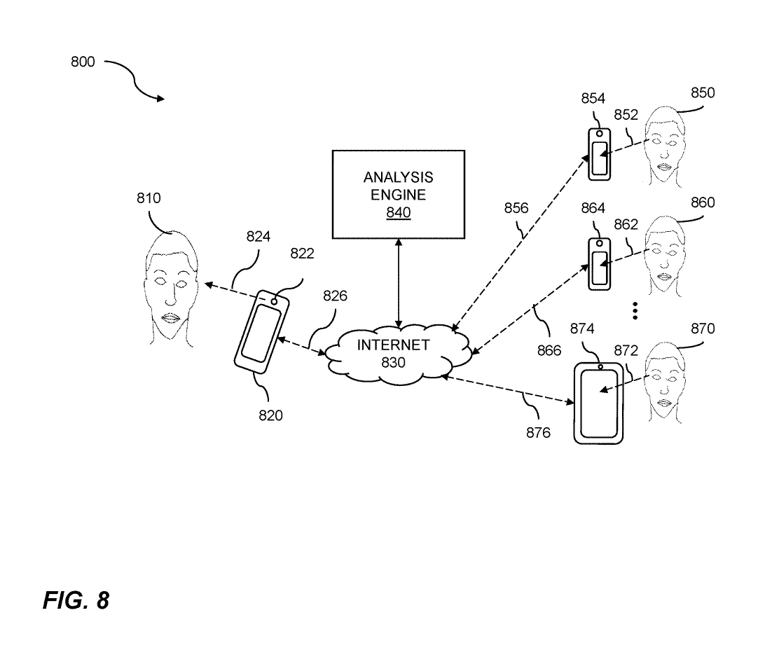

FIG. 8 shows live streaming of social video. The living streaming can be used within a deep learning environment. Analysis of live streaming of social video can be performed using data collected from evaluating images to determine a facial expression and/or mental state. A plurality of images of an individual viewing an electronic display can be received. A face can be identified in an image, based on the use of classifiers. The plurality of images can be evaluated to determine facial expressions and/or mental states of the individual. The streaming and analysis can be facilitated by a video capture device, a local server, a remote server, a semiconductor-based logic, and so on. The streaming can be live streaming and can include mental state analysis, mental state event signature analysis, etc. Live streaming video is an example of one-to-many social media, where video can be sent over the Internet from one person to a plurality of people using a social media app and/or Internet-based platform. Live streaming is one of numerous popular techniques used by people who want to disseminate ideas, send information, provide entertainment, share experiences, and so on in real time. Some of the live streams can be scheduled, such as webcasts, online classes, sporting events, news, computer gaming, or video conferences, while others can be impromptu streams that are broadcasted as needed or when desirable. Examples of impromptu live stream videos can range from individuals simply wanting to share experiences with their social media followers, to live coverage of breaking news, emergencies, or natural disasters. The latter coverage is known as mobile journalism and is becoming increasingly common. With this type of coverage, news reporters can use networked, portable electronic devices to provide mobile journalism content to a plurality of social media followers. Such reporters can be quickly and inexpensively deployed as the need or desire arises.

Several live streaming social media apps and platforms can be used for transmitting video. One such video social media app is Meerkat™ that can link with a user's Twitter™ account. Meerkat™ enables a user to stream video using a handheld, networked electronic device coupled to video capabilities. Viewers of the live stream can comment on the stream using tweets that can be seen by and responded to by the broadcaster. Another popular app is Periscope™ that can transmit a live recording from one user to that user's Periscope™ account and other followers. The Periscope™ app can be executed on a mobile device. The user's Periscope™ followers can receive an alert whenever that user begins a video transmission. Another live-stream video platform is Twitch™ that can be used for video streaming of video gaming and broadcasts of various competitions and events.

The example 800 shows a user 810 broadcasting a video live-stream to one or more people as shown by the person 850, the person 860, and the person 870. A portable, network-enabled electronic device 820 can be coupled to a front-side camera 822. The portable electronic device 820 can be a smartphone, a PDA, a tablet, a laptop computer, and so on. The camera 822 coupled to the device 820 can have a line-of-sight view 824 to the user 810 and can capture video of the user 810. The captured video can be sent to a recommendation or analysis engine 840 using a network link 826 to the Internet 830. The network link can be a wireless link, a wired link, and so on. The analysis engine 840 can recommend to the user 810 an app and/or platform that can be supported by the server and can be used to provide a video live stream to one or more followers of the user 810. In the example 800, the user 810 has three followers: the person 850, the person 860, and the person 870. Each follower has a line-of-sight view to a video screen on a portable, networked electronic device. In other embodiments, one or more followers follow the user 810 using any other networked electronic device, including a computer. In the example 800, the person 850 has a line-of-sight view 852 to the video screen of a device 854; the person 860 has a line-of-sight view 862 to the video screen of a device 864, and the person 870 has a line-of-sight view 872 to the video screen of a device 874. The portable electronic devices 854, 864, and 874 can each be a smartphone, a PDA, a tablet, and so on. Each portable device can receive the video stream being broadcasted by the user 810 through the Internet 830 using the app and/or platform that can be recommended by the analysis engine 840. The device 854 can receive a video stream using the network link 856, the device 864 can receive a video stream using the network link 866, the device 874 can receive a video stream using the network link 876, and so on. The network link can be a wireless link, a wired link, a hybrid link, and so on. Depending on the app and/or platform that can be recommended by the analysis engine 840, one or more followers, such as the followers 850, 860, 870, and so on, can reply to, comment on, and otherwise provide feedback to the user 810 using their devices 854, 864, and 874, respectively. In embodiments, mental state and/or facial expression analysis is performed on each follower (850, 860, and 870). An aggregate viewership score of the content generated by the user 810 can be calculated. The viewership score can be used to provide a ranking of the user 810 on a social media platform. In such an embodiment, users that provide more engaging and more frequently viewed content receive higher ratings.

The human face provides a powerful communications medium through its ability to exhibit a myriad of expressions that can be captured and analyzed for a variety of purposes. In some cases, media producers are acutely interested in evaluating the effectiveness of message delivery by video media. Such video media includes advertisements, political messages, educational materials, television programs, movies, government service announcements, etc. Automated facial analysis can be performed on one or more video frames containing a face in order to detect facial action. Based on the facial action detected, a variety of parameters can be determined, including affect valence, spontaneous reactions, facial action units, and so on. The parameters that are determined can be used to infer or predict emotional and mental states. For example, determined valence can be used to describe the emotional reaction of a viewer to a video media presentation or another type of presentation. Positive valence provides evidence that a viewer is experiencing a favorable emotional response to the video media presentation, while negative valence provides evidence that a viewer is experiencing an unfavorable emotional response to the video media presentation. Other facial data analysis can include the determination of discrete emotional states of the viewer or viewers.

Facial data can be collected from a plurality of people using any of a variety of cameras. A camera can include a webcam, a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. In some embodiments, the person is permitted to “opt-in” to the facial data collection. For example, the person can agree to the capture of facial data using a personal device such as a mobile device or another electronic device by selecting an opt-in choice. Opting-in can then turn on the person's webcam-enabled device and begin the capture of the person's facial data via a video feed from the webcam or other camera. The video data that is collected can include one or more persons experiencing an event. The one or more persons can be sharing a personal electronic device or can each be using one or more devices separately for video capture. The videos that are collected can be collected using a web-based framework. The web-based framework can be used to display the video media presentation or event as well as to collect videos from multiple viewers who are online. That is, the collection of videos can be crowdsourced from those viewers who elected to opt-in to the video data collection.

The videos captured from the various viewers who chose to opt-in can be substantially different in terms of video quality, frame rate, etc. As a result, the facial video data can be scaled, rotated, and otherwise adjusted to improve consistency. Human factors further play into the capture of the facial video data. The facial data that is captured might or might not be relevant to the video media presentation being displayed. For example, the viewer might not be paying attention, might be fidgeting, might be distracted by an object or event near the viewer, or otherwise inattentive to the video media presentation. The behavior exhibited by the viewer can prove challenging to analyze due to viewer actions including eating, speaking to another person or persons, speaking on the phone, etc. The videos collected from the viewers might also include other artifacts that pose challenges during the analysis of the video data. The artifacts can include items such as eyeglasses (because of reflections), eye patches, jewelry, and clothing that occludes or obscures the viewer's face. Similarly, a viewer's hair or hair covering can present artifacts by obscuring the viewer's eyes and/or face.

The captured facial data can be analyzed using the facial action coding system (FACS). The FACS seeks to define groups or taxonomies of facial movements of the human face. The FACS encodes movements of individual muscles of the face, where the muscle movements often include slight, instantaneous changes in facial appearance. The FACS encoding is commonly performed by trained observers but can also be performed on automated, computer-based systems. Analysis of the FACS encoding can be used to determine emotions of the persons whose facial data is captured in the videos. The FACS is used to encode a wide range of facial expressions that are anatomically possible for the human face. The FACS encodings include action units (AUs) and related temporal segments that are based on the captured facial expression. The AUs are open to higher order interpretation and decision-making. These AUs can be used to recognize emotions experienced by the observed person. Emotion-related facial actions can be identified using the emotional facial action coding system (EMFACS) and the facial action coding system affect interpretation dictionary (FACSAID). For a given emotion, specific action units can be related to the emotion. For example, the emotion of anger can be related to AUs 4, 5, 7, and 23, while happiness can be related to AUs 6 and 12. Other mappings of emotions to AUs have also been previously associated. The coding of the AUs can include an intensity scoring that ranges from A (trace) to E (maximum). The AUs can be used for analyzing images to identify patterns indicative of a particular mental and/or emotional state. The AUs range in number from 0 (neutral face) to 98 (fast up-down look). The AUs include so-called main codes (inner brow raiser, lid tightener, etc.), head movement codes (head turn left, head up, etc.), eye movement codes (eyes turned left, eyes up, etc.), visibility codes (eyes not visible, entire face not visible, etc.), and gross behavior codes (sniff, swallow, etc.). Emotion scoring can be included where intensity is evaluated, as well as specific emotions, moods, or mental states.

The coding of faces identified in videos captured of people observing an event can be automated. The automated systems can detect facial AUs or discrete emotional states. The emotional states can include amusement, fear, anger, disgust, surprise, and sadness. The automated systems can be based on a probability estimate from one or more classifiers, where the probabilities can correlate with an intensity of an AU or an expression. The classifiers can be used to identify into which of a set of categories a given observation can be placed. In some cases, the classifiers can be used to determine a probability that a given AU or expression is present in a given frame of a video. The classifiers can be used as part of a supervised machine learning technique, where the machine learning technique can be trained using “known good” data. Once trained, the machine learning technique can proceed to classify new data that is captured.

The supervised machine learning models can be based on support vector machines (SVMs). An SVM can have an associated learning model that is used for data analysis and pattern analysis. For example, an SVM can be used to classify data that can be obtained from collected videos of people experiencing a media presentation. An SVM can be trained using “known good” data that is labeled as belonging to one of two categories (e.g. smile and no-smile). The SVM can build a model that assigns new data into one of the two categories. The SVM can construct one or more hyperplanes that can be used for classification. The hyperplane that has the largest distance from the nearest training point can be determined to have the best separation. The largest separation can improve the classification technique by increasing the probability that a given data point can be properly classified.

In another example, a histogram of oriented gradients (HoG) can be computed. The HoG can include feature descriptors and can be computed for one or more facial regions of interest. The regions of interest of the face can be located using facial landmark points, where the facial landmark points can include outer edges of nostrils, outer edges of the mouth, outer edges of eyes, etc. A HoG for a given region of interest can count occurrences of gradient orientation within a given section of a frame from a video, for example. The gradients can be intensity gradients and can be used to describe an appearance and a shape of a local object. The HoG descriptors can be determined by dividing an image into small, connected regions, also called cells. A histogram of gradient directions or edge orientations can be computed for pixels in the cell. Histograms can be contrast-normalized based on intensity across a portion of the image or the entire image, thus reducing any influence from illumination or shadowing changes between and among video frames. The HoG can be computed on the image or on an adjusted version of the image, where the adjustment of the image can include scaling, rotation, etc. The image can be adjusted by flipping the image around a vertical line through the middle of a face in the image. The symmetry plane of the image can be determined from the tracker points and landmarks of the image.

In embodiments, an automated facial analysis system identifies five facial actions or action combinations in order to detect spontaneous facial expressions for media research purposes. Based on the facial expressions that are detected, a determination can be made with regard to the effectiveness of a given video media presentation, for example. The system can detect the presence of the AUs or the combination of AUs in videos collected from a plurality of people. The facial analysis technique can be trained using a web-based framework to crowdsource videos of people as they watch online video content. The video can be streamed at a fixed frame rate to a server. Human labelers can code for the presence or absence of facial actions including a symmetric smile, unilateral smile, asymmetric smile, and so on. The trained system can then be used to automatically code the facial data collected from a plurality of viewers experiencing video presentations (e.g. television programs).

Spontaneous asymmetric smiles can be detected in order to understand viewer experiences. Related literature indicates that as many asymmetric smiles occur on the right hemi face as do on the left hemi face, for spontaneous expressions. Detection can be treated as a binary classification problem, where images that contain a right asymmetric expression are used as positive (target class) samples and all other images as negative (non-target class) samples. Classifiers perform the classification, including classifiers such as support vector machines (SVM) and random forests. Random forests can include ensemble-learning methods that use multiple learning algorithms to obtain better predictive performance. Frame-by-frame detection can be performed to recognize the presence of an asymmetric expression in each frame of a video. Facial points can be detected, including the top of the mouth and the two outer eye corners. The face can be extracted, cropped, and warped into a pixel image of specific dimension (e.g. 96×96 pixels). In embodiments, the inter-ocular distance and vertical scale in the pixel image are fixed. Feature extraction can be performed using computer vision software such as OpenCV™. Feature extraction can be based on the use of HoGs. HoGs can include feature descriptors and can be used to count occurrences of gradient orientation in localized portions or regions of the image. Other techniques can be used for counting occurrences of gradient orientation, including edge orientation histograms, scale-invariant feature transformation descriptors, etc. The AU recognition tasks can also be performed using Local Binary Patterns (LBP) and Local Gabor Binary Patterns (LGBP). The HoG descriptor represents the face as a distribution of intensity gradients and edge directions, and is robust in its ability to translate and scale. Differing patterns, including groupings of cells of various sizes and arranged in variously sized cell blocks, can be used. For example, 4×4 cell blocks of 8×8 pixel cells with an overlap of half of the block can be used. Histograms of channels can be used, including nine channels or bins evenly spread over 0-180 degrees. In this example, the HoG descriptor on a 96×96 image is 25 blocks×16 cells×9 bins=3600, the latter quantity representing the dimension. AU occurrences can be rendered. The videos can be grouped into demographic datasets based on nationality and/or other demographic parameters for further detailed analysis. This grouping and other analyses can be facilitated via semiconductor-based logic.

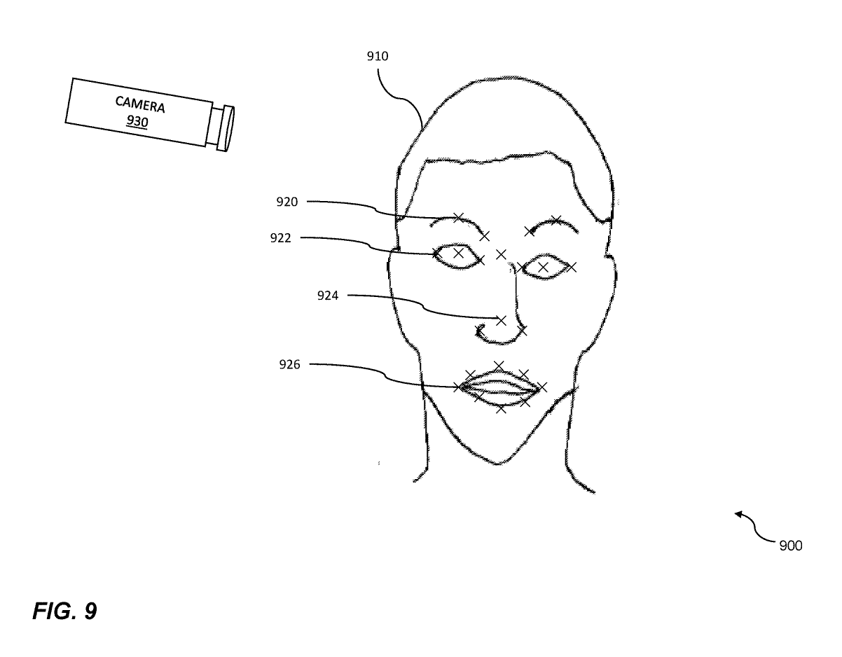

FIG. 9 shows example facial data collection including landmarks. The landmarks can be evaluated by a multilayer analysis system. The collecting of facial data including landmarks can be performed for images that have been collected of an individual. The collected images can be analyzed for mental states and/or facial expressions. A plurality of images of an individual viewing an electronic display can be received. A face can be identified in an image, based on the use of classifiers. The plurality of images can be evaluated to determine mental states and/or facial expressions of the individual. In the example 900, facial data including facial landmarks can be collected using a variety of electronic hardware and software techniques. The collecting of facial data including landmarks can be based on sub-sectional components of a population. The sub-sectional components can be used with performing the evaluation of content of the face, identifying facial landmarks, etc. The sub-sectional components can be used to provide a context. A face 910 can be observed using a camera 930 in order to collect facial data that includes facial landmarks. The facial data can be collected from a plurality of people using one or more of a variety of cameras. As previously discussed, the camera or cameras can include a webcam, where a webcam can include a video camera, a still camera, a thermal imager, a CCD device, a smartphone camera, a three-dimensional camera, a depth camera, a light field camera, multiple webcams used to show different views of a person from various angles, or any other type of image capture apparatus that can allow captured data to be used in an electronic system. The quality and usefulness of the facial data that is captured can depend on the position of the camera 930 relative to the face 910, the number of cameras used, the illumination of the face, etc. In some cases, if the face 910 is poorly lit or over-exposed (e.g. in an area of overly bright light), the processing of the facial data to identify facial landmarks might be rendered more difficult. In another example, the camera 930 being positioned to the side of the person might prevent capture of the full face. Artifacts can degrade the capture of facial data. For example, the person's hair, prosthetic devices (e.g. glasses, an eye patch, and eye coverings), jewelry, and clothing can partially or completely occlude or obscure the person's face. Data relating to various facial landmarks can include a variety of facial features. The facial features can comprise an eyebrow 920, an outer eye edge 922, a nose 924, a corner of a mouth 926, and so on. Multiple facial landmarks can be identified from the facial data that is captured. The facial landmarks that are identified can be analyzed to identify facial action units. The action units that can be identified can include AU02 outer brow raiser, AU14 dimpler, AU17 chin raiser, and so on. Multiple action units can be identified. The action units can be used alone and/or in combination to infer one or more mental states and emotions. A similar process can be applied to gesture analysis (e.g. hand gestures) with all of the analysis being accomplished or augmented by a mobile device, a server, semiconductor-based logic, and so on.

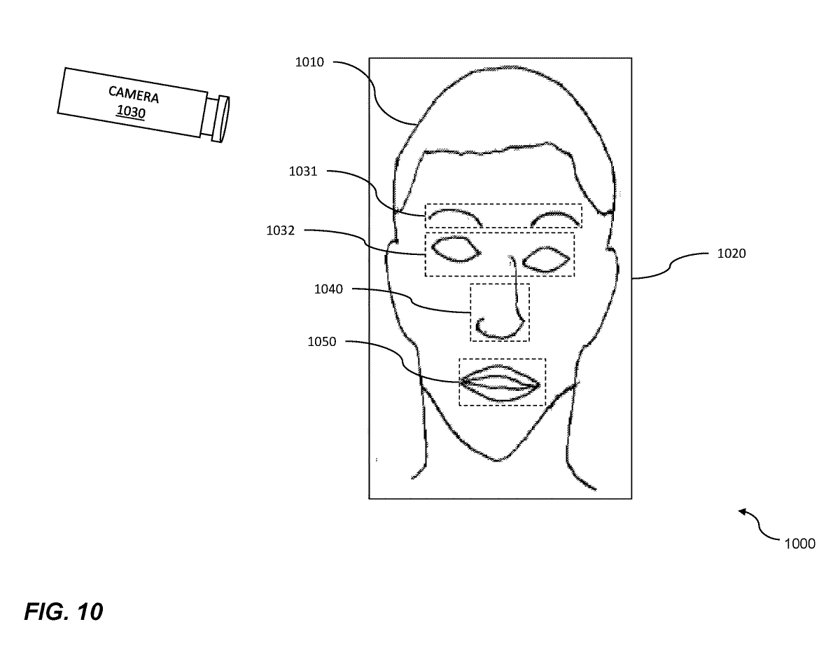

FIG. 10 shows example facial data collection including regions. The regions can be evaluated within a deep learning environment. The collecting of facial data including regions can be performed for images collected of an individual. The collected images can be analyzed for mental states and/or facial expressions. A plurality of images of an individual viewing an electronic display can be received. A face can be identified in an image based on the use of classifiers. The plurality of images can be evaluated to determine mental states and/or facial expressions of the individual. Various regions of a face can be identified and used for a variety of purposes including facial recognition, facial analysis, and so on. The collecting of facial data including regions can be based on sub-sectional components of a population. The sub-sectional components can be used with performing the evaluation of content of the face, identifying facial regions, etc. The sub-sectional components can be used to provide a context. Facial analysis can be used to determine, predict, and estimate mental states and emotions of a person from whom facial data can be collected.

In embodiments, the one or more emotions that can be determined by the analysis can be represented by an image, a figure, an icon, etc. The representative icon can include an emoji or emoticon. One or more emoji can be used to represent a mental state, emotion, or mood of an individual; to represent food, a geographic location, weather, and so on. The emoji can include a static image. The static image can be a predefined size such as a certain number of pixels. The emoji can include an animated image. The emoji can be based on a GIF or another animation standard. The emoji can include a cartoon representation. The cartoon representation can be any cartoon type, format, etc. that can be appropriate to representing an emoji. In the example 1000, facial data can be collected, where the facial data can include regions of a face. The facial data that is collected can be based on sub-sectional components of a population. When more than one face can be detected in an image, facial data can be collected for one face, some faces, all faces, and so on. The facial data which can include facial regions can be collected using any of a variety of electronic hardware and software techniques. The facial data can be collected using sensors including motion sensors, infrared sensors, physiological sensors, imaging sensors, and so on. A face 1010 can be observed using a camera 1030, a sensor, a combination of cameras and/or sensors, and so on. The camera 1030 can be used to collect facial data that can be used to determine if a face is present in an image. When a face is determined to be present in an image, a bounding box 1020 can be placed around the face. Placement of the bounding box around the face can be based on detection of facial landmarks. The camera 1030 can be used to collect facial data from the bounding box 1020, where the facial data can include facial regions. The facial data can be collected from a plurality of people using any of a variety of cameras. As discussed previously, the camera or cameras can include a webcam, where a webcam can include a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. As discussed previously, the quality and usefulness of the facial data that is captured can depend on, among other examples, the position of the camera 1030 relative to the face 1010, the number of cameras and/or sensors used, the level of illumination of the face, any obstructions to viewing the face, and so on.

The facial regions that can be collected by the camera 1030, a sensor, or a combination of cameras and/or sensors can include any of a variety of facial features. Embodiments include determining regions within the face of the individual and evaluating the regions for emotional content. The facial features that can be included in the facial regions that are collected can include eyebrows 1031, eyes 1032, a nose 1040, a mouth 1050, ears, hair, texture, tone, and so on. Multiple facial features can be included in one or more facial regions. The number of facial features that can be included in the facial regions can depend on the desired amount of data to be captured, whether a face is in profile, whether the face is partially occluded or obstructed, etc. The facial regions that can include one or more facial features can be analyzed to determine facial expressions. The analysis of the facial regions can also include determining probabilities of occurrence of one or more facial expressions. The facial features that can be analyzed can also include features such as textures, gradients, colors, and shapes. The facial features can be used to determine demographic data, where the demographic data can include age, ethnicity, culture, and gender. Multiple textures, gradients, colors, shapes, and so on, can be detected by the camera 1030, a sensor, or a combination of cameras and sensors. Texture, brightness, and color, for example, can be used to detect boundaries in an image for detection of a face, facial features, facial landmarks, and so on.

A texture in a facial region can include facial characteristics, skin types, and so on. In some instances, a texture in a facial region can include smile lines, crow's feet, and wrinkles, among others. Another texture that can be used to evaluate a facial region can include a smooth portion of skin such as a smooth portion of a check. A gradient in a facial region can include values assigned to local skin texture, shading, etc. A gradient can be used to encode a texture by computing magnitudes in a local neighborhood or portion of an image. The computed values can be compared to discrimination levels, threshold values, and so on. The gradient can be used to determine gender, facial expression, etc. A color in a facial region can include eye color, skin color, hair color, and so on. A color can be used to determine demographic data, where the demographic data can include ethnicity, culture, age, and gender. A shape in a facial region can include the shape of a face, eyes, nose, mouth, ears, and so on. As with color in a facial region, shape in a facial region can be used to determine demographic data including ethnicity, culture, age, gender, and so on.

The facial regions can be detected based on detection of edges, boundaries, and so on, of features that can be included in an image. The detection can be based on various types of analysis of the image. The features that can be included in the image can include one or more faces. A boundary can refer to a contour in an image plane, where the contour can represent ownership of a particular picture element (pixel) from one object, feature, etc. in the image, to another object, feature, and so on, in the image. An edge can be a distinct, low-level change of one or more features in an image. That is, an edge can be detected based on a change, including an abrupt change such as in color or brightness within an image. In embodiments, image classifiers are used for the analysis. The image classifiers can include algorithms, heuristics, and so on, and can be implemented using functions, classes, subroutines, code segments, etc. The classifiers can be used to detect facial regions, facial features, and so on. As discussed above, the classifiers can be used to detect textures, gradients, color, shapes, and edges, among others. Any classifier can be used for the analysis, including, but not limited to, density estimation, support vector machines (SVM), logistic regression, classification trees, and so on. By way of example, consider facial features that can include the eyebrows 1031. One or more classifiers can be used to analyze the facial regions that can include the eyebrows to determine a probability for either a presence or an absence of an eyebrow furrow. The probability can include a posterior probability, a conditional probability, and so on. The probabilities can be based on Bayesian Statistics or other statistical analysis technique. The presence of an eyebrow furrow can indicate the person from whom the facial data was collected is annoyed, confused, unhappy, and so on. In another example, consider facial features that can include a mouth 1050. One or more classifiers can be used to analyze the facial region that can include the mouth to determine a probability for either a presence or an absence of mouth edges turned up to form a smile. Multiple classifiers can be used to determine one or more facial expressions.

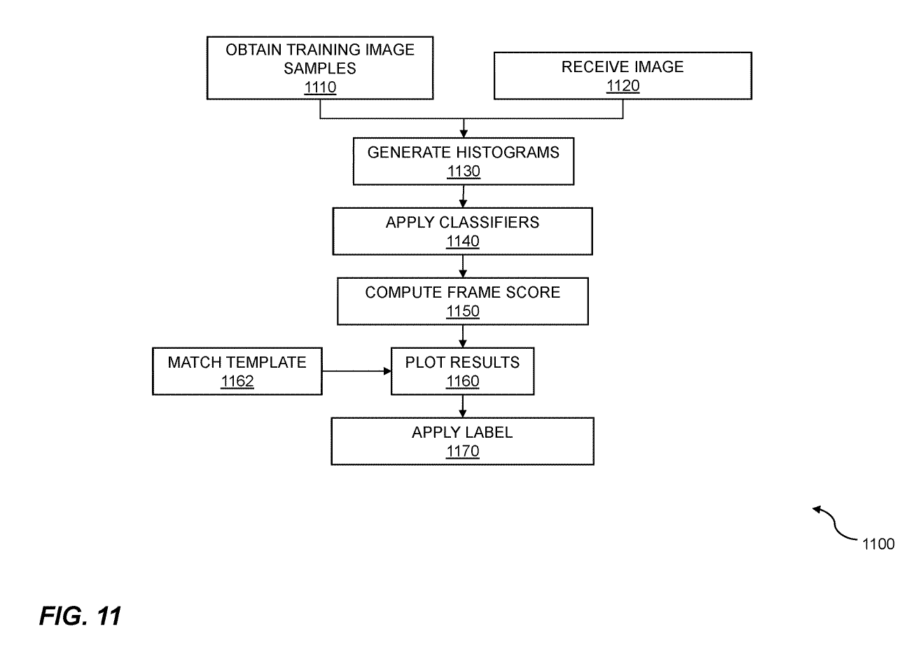

FIG. 11 is a flow diagram for detecting facial expressions. The detection of facial expressions can be performed for data collected from images of an individual and used within a deep learning environment. The collected images can be analyzed for mental states and/or facial expressions. A plurality of images can be received of an individual viewing an electronic display. A face can be identified in an image, based on the use of classifiers. The plurality of images can be evaluated to determine the mental states and/or facial expressions the individual. The flow 1100, or portions thereof, can be implemented in semiconductor logic, can be accomplished using a mobile device, can be accomplished using a server device, and so on. The flow 1100 can be used to automatically detect a wide range of facial expressions. A facial expression can produce strong emotional signals that can indicate valence and discrete emotional states. The discrete emotional states can include contempt, doubt, defiance, happiness, fear, anxiety, and so on. The detection of facial expressions can be based on the location of facial landmarks. The detection of facial expressions can be based on determination of action units (AU), where the action units are determined using FACS coding. The AUs can be used singly or in combination to identify facial expressions. Based on the facial landmarks, one or more AUs can be identified by number and intensity. For example, AU12 can be used to code a lip corner puller and can be used to infer a smirk.

The flow 1100 begins by obtaining training image samples 1110. The image samples can include a plurality of images of one or more people. Human coders who are trained to correctly identify AU codes based on the FACS can code the images. The training, or “known good,” images can be used as a basis for training a machine learning technique. Once trained, the machine learning technique can be used to identify AUs in other images that can be collected using a camera, a sensor, and so on. The flow 1100 continues with receiving an image 1120

The image 1120 can be received from a camera, a sensor, and so on. As previously discussed, the camera or cameras can include a webcam, where a webcam can include a video camera, a still camera, a thermal imager, a CCD device, a smartphone camera, a three-dimensional camera, a depth camera, a light field camera, multiple webcams used to show different views of a person, or any other type of image capture apparatus that can allow captured data to be used in an electronic system. The image that is received can be manipulated in order to improve the processing of the image. For example, the image can be cropped, scaled, stretched, rotated, flipped, etc. in order to obtain a resulting image that can be analyzed more efficiently. Multiple versions of the same image can be analyzed. In some cases, the manipulated image and a flipped or mirrored version of the manipulated image can be analyzed alone and/or in combination to improve analysis. The flow 1100 continues with generating histograms 1130 for the training images and the one or more versions of the received image. The histograms can be based on a HoG or another histogram. As described in previous paragraphs, the HoG can include feature descriptors and can be computed for one or more regions of interest in the training images and the one or more received images. The regions of interest in the images can be located using facial landmark points, where the facial landmark points can include outer edges of nostrils, outer edges of the mouth, outer edges of eyes, etc. A HoG for a given region of interest can count occurrences of gradient orientation within a given section of a frame from a video.

The flow 1100 continues with applying classifiers 1140 to the histograms. The classifiers can be used to estimate probabilities, where the probabilities can correlate with an intensity of an AU or an expression. In some embodiments, the choice of classifiers used is based on the training of a supervised learning technique to identify facial expressions. The classifiers can be used to identify into which of a set of categories a given observation can be placed. The classifiers can be used to determine a probability that a given AU or expression is present in a given image or frame of a video. In various embodiments, the one or more AUs that are present include AU01 inner brow raiser, AU12 lip corner puller, AU38 nostril dilator, and so on. In practice, the presence or absence of multiple AUs can be determined. The flow 1100 continues with computing a frame score 1150. The score computed for an image, where the image can be a frame from a video, can be used to determine the presence of a facial expression in the image or video frame. The score can be based on one or more versions of the image 1120 or a manipulated image. The score can be based on a comparison of the manipulated image to a flipped or mirrored version of the manipulated image. The score can be used to predict a likelihood that one or more facial expressions are present in the image. The likelihood can be based on computing a difference between the outputs of a classifier used on the manipulated image and on the flipped or mirrored image, for example. The classifier that is used can be used to identify symmetrical facial expressions (e.g. smile), asymmetrical facial expressions (e.g. outer brow raiser), and so on.

The flow 1100 continues with plotting results 1160. The results that are plotted can include one or more scores for one or more frames computed over a given time t. For example, the plotted results can include classifier probability results from analysis of HoGs for a sequence of images and video frames. The plotted results can be matched with a template 1162. The template can be temporal and can be represented by a centered box function or another function. A best fit with one or more templates can be found by computing a minimum error. Other best-fit techniques can include polynomial curve fitting, geometric curve fitting, and so on. The flow 1100 continues with applying a label 1170. The label can be used to indicate that a particular facial expression has been detected in the one or more images or video frames which constitute the image 1120 that was received. The label can be used to indicate that any of a range of facial expressions has been detected, including a smile, an asymmetric smile, a frown, and so on. Various steps in the flow 1100 may be changed in order, repeated, omitted, or the like without departing from the disclosed concepts. Various embodiments of the flow 1100 can be included in a computer program product embodied in a non-transitory computer readable medium that includes code executable by one or more processors. Various embodiments of the flow 1100, or portions thereof, can be included on a semiconductor chip and implemented in special purpose logic, programmable logic, and so on.

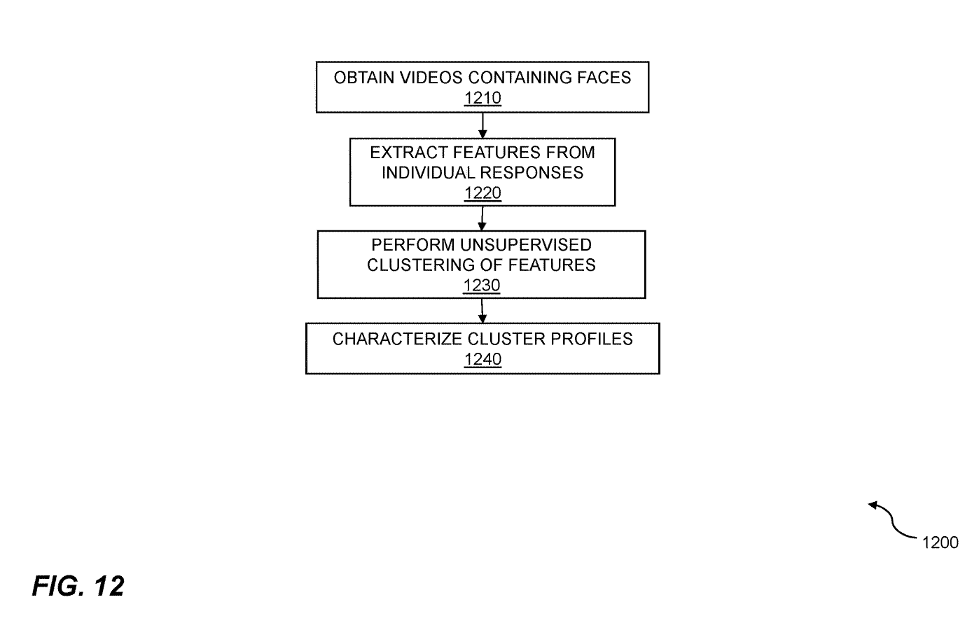

FIG. 12 is a flow diagram for the large-scale clustering of facial events. The large-scale clustering of facial events can be performed for data collected from images of an individual. The collected images can be analyzed for mental states and/or facial expressions. A plurality of images can be received of an individual viewing an electronic display. A face can be identified in an image, based on the use of classifiers. The plurality of images can be evaluated to determine the mental states and/or facial expressions of the individual. The clustering and evaluation of facial events can be augmented using a mobile device, a server, semiconductor-based logic, and so on. As discussed above, collection of facial video data from one or more people can include a web-based framework. The web-based framework can be used to collect facial video data from large numbers of people located over a wide geographic area. The web-based framework can include an opt-in feature that allows people to agree to facial data collection. The web-based framework can be used to render and display data to one or more people and can collect data from the one or more people. For example, the facial data collection can be based on showing one or more viewers a video media presentation through a website. The web-based framework can be used to display the video media presentation or event and to collect videos from multiple viewers who are online. That is, the collection of videos can be crowdsourced from those viewers who elected to opt-in to the video data collection. The video event can be a commercial, a political ad, an educational segment, and so on.

The flow 1200 begins with obtaining videos containing faces 1210. The videos can be obtained using one or more cameras, where the cameras can include a webcam coupled to one or more devices employed by the one or more people using the web-based framework. The flow 1200 continues with extracting features from the individual responses 1220. The individual responses can include videos containing faces observed by the one or more webcams. The features that are extracted can include facial features such as an eyebrow, a nostril, an eye edge, a mouth edge, and so on. The feature extraction can be based on facial coding classifiers, where the facial coding classifiers output a probability that a specified facial action has been detected in a given video frame. The flow 1200 continues with performing unsupervised clustering of features 1230. The unsupervised clustering can be based on an event. The unsupervised clustering can be based on a K-Means, where the K of the K-Means can be computed using a Bayesian Information Criterion (BICk), for example, to determine the smallest value of K that meets system requirements. Any other criterion for K can be used. The K-Means clustering technique can be used to group one or more events into various respective categories.

The flow 1200 continues with characterizing cluster profiles 1240. The profiles can include a variety of facial expressions such as smiles, asymmetric smiles, eyebrow raisers, eyebrow lowerers, etc. The profiles can be related to a given event. For example, a humorous video can be displayed in the web-based framework and the video data of people who have opted-in can be collected. The characterization of the collected and analyzed video can depend in part on the number of smiles that occurred at various points throughout the humorous video. The number of smiles resulting from people viewing a humorous video can be compared to various demographic groups, where the groups can be formed based on geographic location, age, ethnicity, gender, and so on. Similarly, the characterization can be performed on collected and analyzed videos of people viewing a news presentation. The characterized cluster profiles can be further analyzed based on demographic data. Various steps in the flow 1200 may be changed in order, repeated, omitted, or the like without departing from the disclosed concepts. Various embodiments of the flow 1200 can be included in a computer program product embodied in a non-transitory computer readable medium that includes code executable by one or more processors. Various embodiments of the flow 1200, or portions thereof, can be included on a semiconductor chip and implemented in special purpose logic, programmable logic, and so on.

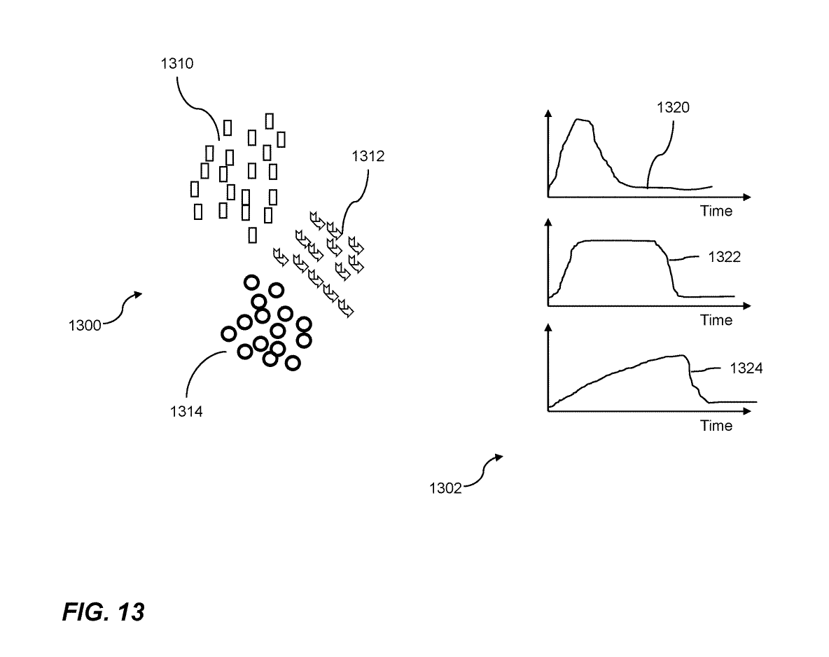

FIG. 13 shows unsupervised clustering of features and characterizations of cluster profiles. The clustering can be accomplished as part of a deep learning effort. The clustering of features and characterizations of cluster profiles can be performed for images collected of an individual. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. Features including samples of facial data can be clustered using unsupervised clustering. Various clusters can be formed which include similar groupings of facial data observations. The example 1300 shows three clusters, clusters 1310, 1312, and 1314. The clusters can be based on video collected from people who have opted-in to video collection. When the data collected is captured using a web-based framework, the data collection can be performed on a grand scale, including hundreds, thousands, or even more participants who can be located locally and/or across a wide geographic area. Unsupervised clustering is a technique that can be used to process the large amounts of captured facial data and to identify groupings of similar observations. The unsupervised clustering can also be used to characterize the groups of similar observations. The characterizations can include identifying behaviors of the participants. The characterizations can be based on identifying facial expressions and facial action units of the participants. Some behaviors and facial expressions can include faster or slower onsets, faster or slower offsets, longer or shorter durations, etc. The onsets, offsets, and durations can all correlate to time. The data clustering that results from the unsupervised clustering can support data labeling. The labeling can include FACS coding. The clusters can be partially or totally based on a facial expression resulting from participants viewing a video presentation, where the video presentation can be an advertisement, a political message, educational material, a public service announcement, and so on. The clusters can be correlated with demographic information, where the demographic information can include educational level, geographic location, age, gender, income level, and so on.

The cluster profiles 1302 can be generated based on the clusters that can be formed from unsupervised clustering, with time shown on the x-axis and intensity or frequency shown on the y-axis. The cluster profiles can be based on captured facial data including facial expressions. The cluster profile 1320 can be based on the cluster 1310, the cluster profile 1322 can be based on the cluster 1312, and the cluster profile 1324 can be based on the cluster 1314. The cluster profiles 1320, 1322, and 1324 can be based on smiles, smirks, frowns, or any other facial expression. The emotional states of the people who have opted-in to video collection can be inferred by analyzing the clustered facial expression data. The cluster profiles can be plotted with respect to time and can show a rate of onset, a duration, and an offset (rate of decay). Other time-related factors can be included in the cluster profiles. The cluster profiles can be correlated with demographic information, as described above.

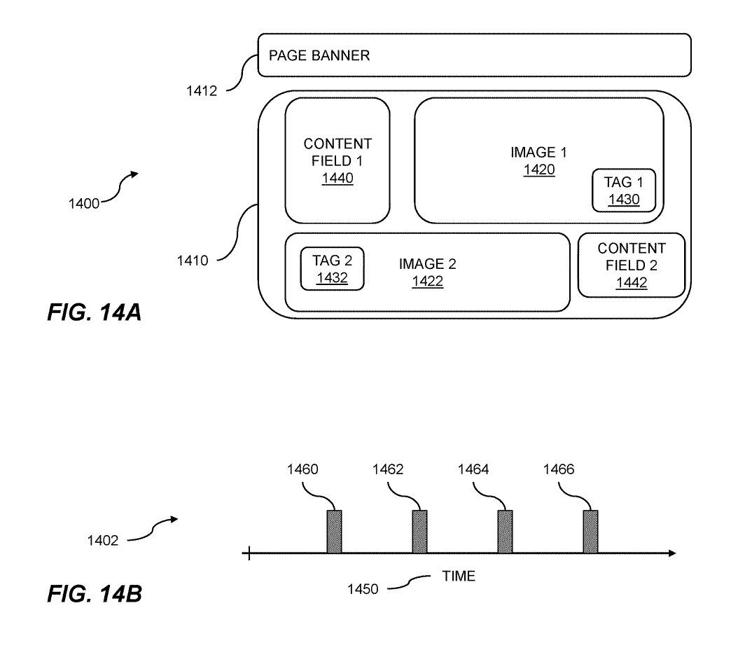

FIG. 14A shows example tags embedded in a webpage. The tags embedded in the webpage can be used for image analysis for images collected of an individual, and the image analysis can be performed by a multilayer system. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. Once a tag is detected, a mobile device, a server, semiconductor-based logic, etc. can be used to evaluate associated facial expressions. A webpage 1400 can include a page body 1410, a page banner 1412, and so on. The page body can include one or more objects, where the objects can include text, images, videos, audio, and so on. The example page body 1410 shown includes a first image, image 1 1420; a second image, image 2 1422; a first content field, content field 1 1440; and a second content field, content field 2 1442. In practice, the page body 1410 can contain multiple images and content fields, and can include one or more videos, one or more audio presentations, and so on. The page body can include embedded tags, such as tag 1 1430 and tag 2 1432. In the example shown, tag 1 1430 is embedded in image 1 1420, and tag 2 1432 is embedded in image 2 1422. In embodiments, multiple tags are embedded. Tags can also be embedded in content fields, in videos, in audio presentations, etc. When a user mouses over a tag or clicks on an object associated with a tag, the tag can be invoked. For example, when the user mouses over tag 1 1430, tag 1 1430 can then be invoked. Invoking tag 1 1430 can include enabling a camera coupled to a user's device and capturing one or more images of the user as the user views a media presentation (or digital experience). In a similar manner, when the user mouses over tag 2 1432, tag 2 1432 can be invoked. Invoking tag 2 1432 can also include enabling the camera and capturing images of the user. In other embodiments, other actions are taken based on invocation of the one or more tags. Invoking an embedded tag can initiate an analysis technique, post to social media, award the user a coupon or another prize, initiate mental state analysis, perform emotion analysis, and so on.

FIG. 14B shows invoking tags to collect images. The invoking tags to collect images can be used for image analysis for images collected of an individual. The collected images can be analyzed for mental states and/or facial expressions. A plurality of images can be received of an individual viewing an electronic display. A face can be identified in an image, based on the use of classifiers. The plurality of images can be evaluated to determine mental states and/or facial expressions of the individual. As previously stated, a media presentation can be a video, a webpage, and so on. A video 1402 can include one or more embedded tags, such as a tag 1460, another tag 1462, a third tag 1464, a fourth tag 1466, and so on. In practice, multiple tags can be included in the media presentation. The one or more tags can be invoked during the media presentation. The collection of the invoked tags can occur over time, as represented by a timeline 1450. When a tag is encountered in the media presentation, the tag can be invoked. When the tag 1460 is encountered, invoking the tag can enable a camera coupled to a user device and can capture one or more images of the user viewing the media presentation. Invoking a tag can depend on a user agreeing to opt-in. For example, if a user has agreed to participate in a study by indicating an opt-in, then the camera coupled to the user's device can be enabled and one or more images of the user can be captured. If the user has not agreed to participate in the study and has not indicated an opt-in, then invoking the tag 1460 does not enable the camera nor capture images of the user during the media presentation. The user can indicate an opt-in for certain types of participation, where opting-in can be dependent on specific content in the media presentation. The user could opt-in to participation in a study of political campaign messages and not opt-in for a particular advertisement study. In this case, tags that are related to political campaign messages, advertising messages, social media sharing, etc. and that enable the camera and image capture when invoked would be embedded in the media presentation, social media sharing, and so on. However, tags embedded in the media presentation that are related to advertisements would not enable the camera when invoked. Various other situations of tag invocation are possible.

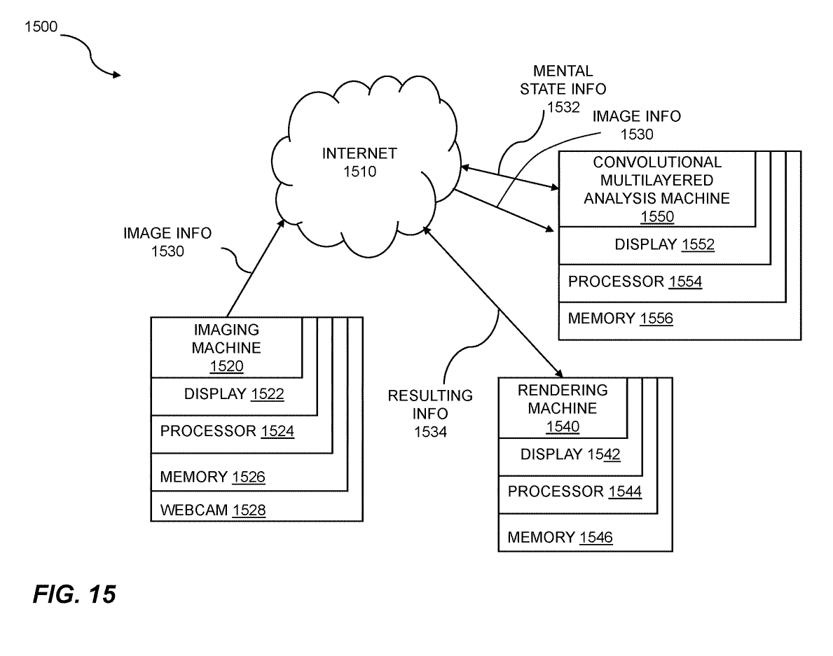

FIG. 15 is a system diagram for image analysis. The system 1500 can include one or more imaging machines 1520 linked to a convolutional multilayered analysis machine 1550 and a rendering machine 1540 via the Internet 1510 or another computer network. The network can be wired or wireless, a combination of wired and wireless networks, and so on. Image information 1530 can be transferred to the convolutional multilayered analysis machine 1550 through the Internet 1510. The example imaging machine 1520 shown comprises one or more processors 1524 coupled to a memory 1526 which can store and retrieve instructions, a display 1522, and a camera 1528. The camera 1528 can include a webcam, a video camera, a still camera, a thermal imager, a CCD device, a smartphone camera, a three-dimensional camera, a depth camera, a light field camera, multiple webcams used to show different views of a person, or any other type of image capture technique that can allow captured data to be used in an electronic system. The memory 1526 can be used for storing instructions, image data on a plurality of people, one or more classifiers, one or more action units, and so on. The display 1522 can be any electronic display, including but not limited to, a computer display, a laptop screen, a net-book screen, a tablet computer screen, a smartphone display, a mobile device display, a remote with a display, a television, a projector, or the like. Mental state information 1532 can be transferred via the Internet 1510 for a variety of purposes including analysis, rendering, storage, cloud storage, sharing, social sharing, and so on.

The convolutional multilayered analysis machine 1550 can include one or more processors 1554 coupled to a memory 1556 which can store and retrieve instructions, and it can also include a display 1552. The convolutional multilayered analysis machine 1550 can receive mental state information 1532 and image information 1530 and analyze the information using classifiers, action units, and so on. The classifiers and action units can be stored in the multilayered analysis machine, loaded into the multilayered analysis machine, provided by a user of the multilayered analysis machine, and so on. The convolutional multilayered analysis machine 1550 can use image data received from the imaging machine 1520 to produce resulting information 1534. The resulting information can include analysis of facial expressions, mood, mental state, etc., and can be based on the image information 1530. In some embodiments, the convolutional multilayered analysis machine 1550 receives image data from a plurality of imaging machines, aggregates the image data, processes the image data or the aggregated image data, and so on.

The rendering machine 1540 can include one or more processors 1544 coupled to a memory 1546 which can store and retrieve instructions and data, and it can also include a display 1542. The rendering of the resulting information 1534 can occur on the rendering machine 1540 or on a different platform from the rendering machine 1540. In embodiments, the rendering of the resulting information rendering data occurs on the imaging machine 1520 or on the convolutional multilayered analysis machine 1550. As shown in the system 1500, the rendering machine 1540 can receive resulting information 1534 via the Internet 1510 or another network from the imaging machine 1520, from the convolutional multilayered analysis machine 1550, or from both. The rendering can include a visual display or any other appropriate display format.

The system 1500 can include a computer system for image analysis comprising: a memory which stores instructions; one or more processors attached to the memory wherein the one or more processors, when executing the instructions which are stored, are configured to: initialize the computer system for convolutional processing; obtain, using an imaging device, a plurality of images; train, on the computer initialized for convolutional processing, a multilayered analysis engine using the plurality of images, wherein the multilayered analysis engine includes one or more convolutional layers and one or more hidden layers, and wherein the multilayered analysis engine is used for emotional analysis; and evaluate a further image using the multilayered analysis engine wherein the evaluating includes: analyzing pixels within the further image to identify a facial portion; and inferring a mental state based on emotional content within a face associated with the facial portion.

The system 1500 can include a computer program product embodied in a non-transitory computer readable medium for image analysis, the computer program product comprising code which causes one or more processors to perform operations of: initializing a computer for convolutional processing; obtaining, using an imaging device, a plurality of images; training, on the computer initialized for convolutional processing, a multilayered analysis engine using the plurality of images, wherein the multilayered analysis engine includes one or more convolutional layers and one or more hidden layers, and wherein the multilayered analysis engine is used for emotional analysis; and evaluating a further image using the multilayered analysis engine wherein the evaluating includes: analyzing pixels within the further image to identify a facial portion; and inferring a mental state based on emotional content within a face associated with the facial portion.

The system 1500 can include a computer-implemented method for image analysis comprising: initializing a computer for convolutional processing; obtaining, using an imaging device, a plurality of images; training, on the computer initialized for convolutional processing, a multilayered analysis engine using the plurality of images, wherein the multilayered analysis engine includes multiple layers that include one or more convolutional layers and one or more hidden layers, and wherein the multilayered analysis engine is used for emotional analysis; and evaluating a further image using the multilayered analysis engine wherein the evaluating includes: analyzing pixels within the further image to identify a facial portion; and inferring a mental state based on emotional content within a face associated with the facial portion.

The system 1500 can include a computer-implemented method for image analysis comprising: initializing a computer for convolutional processing; obtaining, using an imaging device, a plurality of images; training, on the computer initialized for convolutional processing, a multilayered analysis engine using the plurality of images, wherein the multilayered analysis engine includes multiple layers that include one or more convolutional layers and one or more hidden layers, and wherein the multilayered analysis engine is used for emotional analysis; and evaluating a further image using the multilayered analysis engine wherein the evaluating includes: analyzing pixels within the further image to identify a facial portion; and identifying a facial expression based on the facial portion.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”— may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for image analysis comprising:
initializing a computer for convolutional processing;
obtaining, using an imaging device, a plurality of images;
training, on the computer initialized for convolutional processing, a multilayered analysis engine using the plurality of images, wherein the multilayered analysis engine includes multiple layers that include one or more convolutional layers and one or more hidden layers, and wherein the multilayered analysis engine is used for emotional analysis; and
evaluating a further image using the multilayered analysis engine wherein the evaluating includes:
analyzing pixels within the further image to identify a facial portion; and
identifying a facial expression based on the facial portion.

2. The method of claim 1 wherein a last layer within the multiple layers provides output indicative of mental state.

3. The method of claim 2 further comprising tuning the last layer within the multiple layers for a particular mental state.

4. The method of claim 1 wherein the multilayered analysis engine further includes a max pooling layer.

5. The method of claim 1 wherein the training comprises assigning weights to inputs on one or more layers within the multilayered analysis engine.

6. The method of claim 5 wherein the assigning weights is accomplished during a feed-forward pass through the multilayered analysis engine.

7. The method of claim 6 wherein the weights are updated during a backpropagation process through the multilayered analysis engine.

8. The method of claim 1 further comprising rotating a face within the plurality of images.

9. The method of claim 1 further comprising performing supervised learning as part of the training by using a set of images, from the plurality of images, that have been labeled for mental states.

10. The method of claim 1 further comprising performing unsupervised learning as part of the training.

11. (canceled)

12. The method of claim 1 further comprising learning image descriptors, as part of the training, for emotional content.

13. The method of claim 12 wherein the image descriptors are identified based on a temporal co-occurrence with an external stimulus.

14. The method of claim 1 further comprising training an emotion classifier, as part of the training, for emotional content.

15. The method of claim 1 wherein the training of the multilayered analysis engine comprises deep learning.

16. The method of claim 1 wherein the multilayered analysis engine comprises a convolutional neural network.

17. The method of claim 1 further comprising re-training the multilayered analysis engine using a second plurality of images.

18. The method of claim 17 wherein the re-training updates weights on a subset of layers within the multilayered analysis engine.

19. The method of claim 18 wherein the subset of layers is a single layer within the multilayered analysis engine.

20. The method of claim 1 further comprising inferring a mental state based on emotional content within a face associated with the facial portion.

21. The method of claim 20 wherein the facial expression is identified using a hidden layer from the one or more hidden layers.

22. The method of claim 20 wherein weights are provided on inputs to the multiple layers to emphasize certain facial features within the face.

23. The method of claim 20 further comprising identifying boundaries of the face.

24. The method of claim 20 further comprising identifying landmarks of the face.

25. The method of claim 20 further comprising extracting features of the face.

26. The method of claim 20 wherein inferring a mental state based on emotional content within the face includes detection of one or more of sadness, stress, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, curiosity, humor, sadness, poignancy, or mirth.

27. A computer-implemented method for image analysis comprising:
initializing a computer for convolutional processing;
obtaining, using an imaging device, a plurality of images;
training, on the computer initialized for convolutional processing, a multilayered analysis engine using the plurality of images, wherein the multilayered analysis engine includes multiple layers that include one or more convolutional layers and one or more hidden layers, and wherein the multilayered analysis engine is used for emotional analysis; and
evaluating a further image using the multilayered analysis engine wherein the evaluating includes:
analyzing pixels within the further image to identify a facial portion; and
inferring a mental state based on emotional content within a face associated with the facial portion.

28-29. (canceled)
